(19) United States
(12) Patent Application Publication
(10) Pub. No.: US 2025/0073248 A1
(43) Pub. Date: Mar. 6, 2025

(54) BIORESORBABLE CATIONIC STEROIDAL ANTIMICROBIAL COMPOUNDS WITH GLYCERIDE LINKAGES

(71) Applicant: Paul B. SAVAGE, Mapleton, UT (US)

(72) Inventor: Paul B. SAVAGE, Mapleton, UT (US)

(73) Assignee: BRIGHAM YOUNG UNIVERSITY, Provo, UT (US)

(21) Appl. No.: 18/823,369

(22) Filed: Sep. 3, 2024

Related U.S. Application Data
(60) Provisional application No. 63/536,613, filed on Sep. 5, 2023.

(51) Int. Cl. A61K 31/575; A61P 31/00

(52) U.S. Cl. CPC A61K 31/575; A61P 31/00

(57) ABSTRACT

Bioresorbable cationic steroidal antimicrobial (CSA) compounds having a structure of Formula I, II or III, or salt thereof, wherein at least one of R₁-R₁₈, (e.g., R₁₈) includes a mono-or diglyceride of a fatty acid attached via an ester linkage and at least one R₁-R₁₈, (e.g., R₃, R₇ and R₁₂) is an amino acid linked to the steroidal backbone via an ester or amide linkage:

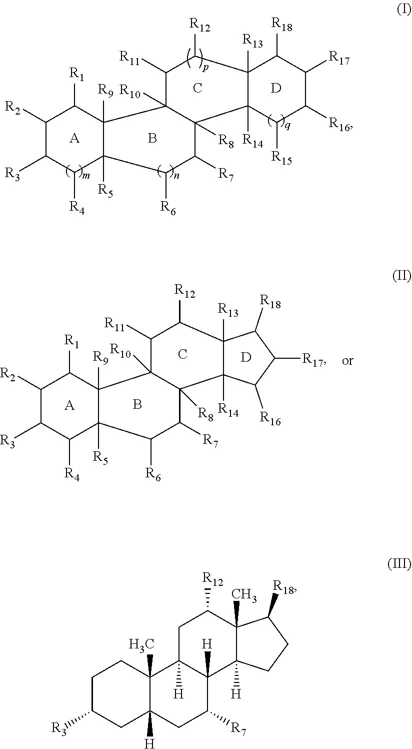

wherein:
R₁₈ has the following structure:

—R₁₉—(C═O)—O—C—CR₂₀—CR₂₁

where R₁₉ is omitted or selected from alkyl, alkenyl, alkynyl, and aryl, and R₂₀ and R₂₁ are independently selected from hydroxy, alkylcarboxy, and aminoalkylcarboxy, provided that at least one of R₂₀ or R₂₁ is an alkylcarboxy,
R₃, R₇ and R₁₂ have the following structure:

R₂₄R₂₃N—R₂₂—(C═O)—X—

where R₂₂ is substituted or unsubstituted alkyl, X is oxygen or nitrogen, and R₂₃ and R₂₄ are independently hydrogen, alkyl, alkenyl, alkynyl, or aryl.

### CROSS-REFERENCE TO RELATED APPLICATIONS

This application claims the benefit of U.S. Provisional Application No. 63/536,613, filed Sep. 5, 2024, which is incorporate by reference in its entirety.

### BACKGROUND OF THE INVENTION

### 1. Field

Disclosed are cationic steroidal antimicrobial (CSA) compounds, more particularly bioresorbable CSA compounds with glyceride linkages, more particularly mono-and diglycerides glycerides of CSA compounds and fatty acids, and methods of manufacturing bioresorbable glycerides of CSA compounds.

### 2. Related Technology

Hospital acquired infections (HAIs) are a subset of infections leading to more than 98,000 deaths per year globally. As many as 70% of all HAIs are associated with in-dwelling medical devices and are collectively reported as device related infections (DRIs). Costs associated with treating DRIs are substantial, and decreasing DRIs would not only decrease mortality from HAIs but also reduce billions of dollars spent yearly combatting them. DRIs are generally associated with bacterial and/or fungal biofilms growing on medical devices. Microbial biofilms are communities of organisms, often polymicrobial, in which organisms can become sessile and highly resistant to most antimicrobial agents. Furthermore, the proximity of organisms within biofilms promotes genetic transfer leading to exchange of drug-resistance mechanisms.

Development of medical devices that can prevent microbes from growing on their surfaces would necessarily improve patient outcomes. An attractive approach to prevent microbial biofilm formation on medical devices involves elution of an antimicrobial agent from the surface of the device. Potential issues with eluting antimicrobial agents from the surfaces of medical devices is local toxicity to host cells and systemic exposure to the antimicrobial. As an antimicrobial enters systemic distribution, the natural flora may be disturbed and repeated exposure to an antimicrobial can lead to generation of resistance.

A possible means of circumventing these issues would be a broad-spectrum antimicrobial that acts on or near the surface of medical devices while being rapidly degraded to endogenous compounds upon elution from the device. Bioresorbable materials are already commonly used in medical care and degrade slowly in the presence of water. In general, these materials spontaneously degrade to yield either endogenous compounds or compounds that can be converted into endogenous compounds through enzymatic activity. Few bioresorbable materials have been developed that have broad spectrum antimicrobial effects at low concentrations.

Most tissues in higher organisms continuously produce natural antimicrobial compounds, including antimicrobial peptides (AMPs), as a means of controlling microbial growth. Providing similar protection to medical devices would be an attractive goal. AMPs are bioresorbable and are resorbed after proteolytic cleavage. Although bacteria and fungi have been exposed to AMPs over eons of time, they remain, in general, susceptible to AMPs and have not built up resistance.

In humans, the AMP LL-37 (a member of the cathelicidin family) has been well characterized as an antimicrobial agent with activities against Gram-positive and Gram-negative bacteria, fungi and lipid-enveloped viruses. LL-37 is found in most human tissues, including the skin, gastrointestinal tract lining, the lungs, and even on the surface of the eyes. Cathelicidin LL-37 selectively associates with microbial membranes and causes an increase in membrane permeability leading to microbial cell death. AMP resistance is infrequent and requires bacteria and other microbes to alter their membrane structure.

The ubiquity of AMPs has been used as evidence that these compounds do not readily engender bacterial resistance. In addition, considering the varied sequences of antimicrobial peptides among diverse organisms, it is apparent that they have evolved independently multiple times. Thus, antimicrobial peptides appear to be one of “Nature's” primary means of controlling bacterial growth. For example, endogenous antimicrobial peptides, such as the human cathelicidin LL-37, play key roles in innate immunity. LL-37 is found in airway mucus and is believed to be important in controlling bacterial growth in the lung. However, clinical use of AMPs presents significant issues, including the high cost of producing peptide-based therapeutics, the susceptibility of peptides to proteases generated by the host and by bacterial pathogens, and deactivation of antimicrobial peptides by proteins and DNA in lung mucosa.

An attractive way of harnessing the antibacterial activities of antimicrobial peptides without the issues delineated above would be to develop non-peptide mimics of antimicrobial peptides that display similar broad-spectrum antibacterial activity utilizing the same or similar mechanism of action. Non-peptide mimics would offer lower-cost synthesis and potentially increased stability to proteolytic degradation. In addition, control of water solubility and charge density may be used to control association with proteins and DNA in lung mucosa.

With over 1,600 examples of known antimicrobial peptides, it is possible to categorize the structural features common to them. While the primary sequences of these peptides vary substantially, morphologies adopted by a vast majority are similar. Those that adopt alpha helix conformations juxtapose hydrophobic side chains on one face of the helix with cationic (positively charged) side chains on the opposite side. Similar morphology is found in antimicrobial peptides that form beta sheet structures: hydrophobic side chains on one face of the sheet and cationic side chains on the other.

Examples of small molecule, non-peptide mimics of antimicrobial peptides, include steroidal compounds known as “ceragenins”, “CSAs” and “CSA compounds”, examples of which are “CSA-13” and “CSA-44”, which can reproduce the amphiphilic morphology in antimicrobial peptides. A problem that remains is that CSA compounds have side groups that form non-endogenous degradation products that may not be approved for the human body, which are not entirely safe at higher concentrations, and which are not fully bioresorbable. Another problem is that CSA compounds often require complex, multi-step reaction sequences for their manufacture. Every additional step in the manufacture of CSA compounds increases cost and reduces overall product yield.

Accordingly, there remains a need to find improved CSA compounds that can provide desired antimicrobial properties but which are fully bioresorbable, safe at higher concentrations, and relatively easy to manufacture.

### SUMMARY

Disclosed herein is a new class of cationic steroidal antimicrobial (CSA) compounds with bioresorbable groups attached to a bioresorbable sterol backbone via one or more ester or other hydrolysable linkages, include mono- and diglyceride linkages. This class of CSA compounds is referred to as “bioresorbable CSA compounds”.

The degradation products of bioresorbable CSA compounds are themselves bioresorbable, such as cholic acid (a common bile acid), amino acids (e.g., beta-alanine, a bioresorbable neurotransmitter and amino acid), glycerin, and fatty acids (e.g., short-chain, medium-chain, and long-chain fatty acids). The bioresorbable CSA compounds are relatively easy and inexpensive to manufacture, are safe at higher concentrations, and possess desired antimicrobial, anti-inflammatory, and other desirable properties.

The bioresorbable CSA compounds disclosed herein can have a structure of Formula I, II or III, or a salt thereof, having a steroidal backbone, and wherein at least one of R₁-R₁₈, preferably R₁₈, can include a glyceride group and at least one fatty acid, such as butyric acid, caproic acid, caprylic acid, capric acid, lauric acid, or myristic acid, at the C24 position of the steroidal backbone, and at least one of R₁-R₁₈, preferably at least one of R₃, R₇ and R₁₂, can comprise an amino acid, e.g., beta-alanine, linked to the sterol backbone by an ester linkage at the C3, C7, and/or C12 positions(s), and optionally the C24 position of R₁₈:

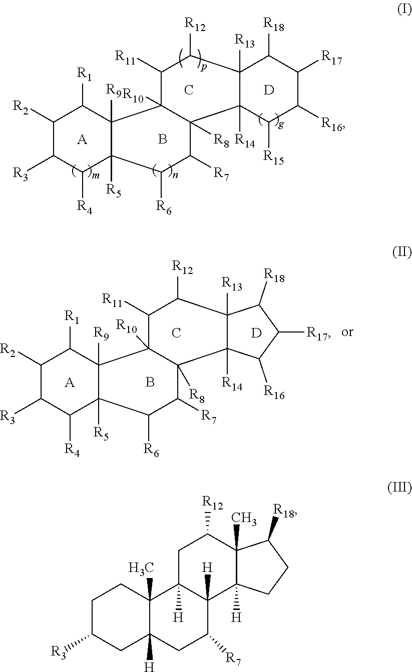

where R₁₅ is omitted in Formulas II and III.

In some embodiments, at least one of R₁-R₁₈, preferably R₁₈, can have the following mono-or diglyceride structure:

—R₁₉—(C═O)—O—C—CR₂₀—CR₂₁

where R₁₉ is omitted or is selected from alkyl, alkenyl, alkynyl, and aryl, and R₂₀ and R₂₁ are independently selected from hydroxy and (C₂-C₂₂)alkylcarboxy, provided that at least one of R₂₀ or R₂₁ is (C₂-C₂₂)alkylcarboxy, the (C₂-C₂₂)alkylcarboxy preferably having an even number of carbons. The glyceride portion of foregoing structure forms bioresorbable glycerin and fatty acid(s) as degradation product (e.g., by hydrolysis of ester groups in the glyceride structure).

In some embodiments, R₁₈ can have the following mixed diglyceride structure:

—R₁₉—(C═O)—O—C—CR₂₀—CR₂₁

where R₁₉ is omitted or is selected from alkyl, alkenyl, alkynyl, and aryl, R₂₀ is a (C₂-C₂₂)alkylcarboxy, the (C₂-C₂₂)alkylcarboxy preferably having an even number of carbons, and R₂₁ can have the following aminoalkylcarboxy structure:

R₂₄R₂₃N—R₂₂—(C═O)—O—

where R₂₂ is a substituted or unsubstituted alkyl and R₂₃ and R₂₄ are independently selected from hydrogen, alkyl, alkenyl, alkynyl, and aryl. R₂₂ is preferably an ester group of an amino acid, such as beta-alanine, which forms a bioresorbable amino acid (e.g., beta-alanine) as degradation product (e.g., by hydrolysis of the ester groups at the C24). The glyceride portion forms bioresorbable glycerin, a fatty acid, and an amino acid as degradation products (e.g., by hydrolysis of ester groups in the glyceride structure).

In some embodiments, at least one of R₁-R₁₈, preferably at least one of R₃, R₇ and R₁₂, can have the following aminoalkylcarboxy or aminoalkylcarboxamido structure:

R₂₄R₂₃N—R₂₂—(C═O)—X—where

R₂₂ is a substituted or unsubstituted alkyl, X is oxygen or nitrogen, and R₂₃ and R₂₄ are independently selected from hydrogen, alkyl, alkenyl, alkynyl, and aryl. At least one of R₃, R₇and R₁₂, preferably two or three of R₃, R₇ and R₁₂, is/are an ester group of one or more amino acids, such as beta-alanine, which forms a bioresorbable amino acid (e.g., beta-alanine) as degradation product (e.g., by hydrolysis of the ester group(s) at the C3, C7 and/or C12position(s)). Alternatively, the aminoalkyl portion of at least one of R₃, R₇ and R₁₂ can be attached to one or more of the C3, C7 and/or C12 positions of the sterol backbone (or elsewhere) by other linkages, such as amide or ether linkage.

In some embodiments, bioresorbable mono- and diglyceride CSA compounds can have a chiral center, such as in the glyceryl moiety, so as to form enantiomers that can be isolated rather than forming a racemic mixture. Unless otherwise specified, the examples of CSA compounds disclosed herein can be non-chiral, R- and S-enantiomers forming a racemic mixture, the R-enantiomer, or the S-enantiomer.

Non-limiting examples of bioresorbable monoglyceride CSA compounds that form bioresorbable degradation products by hydrolysis of ester groups are CSA-4108, CSA-4110 (racemic mixture), CSA-4110R (R-enantiomer), CSA-4110S (S-enantiomer), CSA-4112, CSA-4114, and salts thereof:

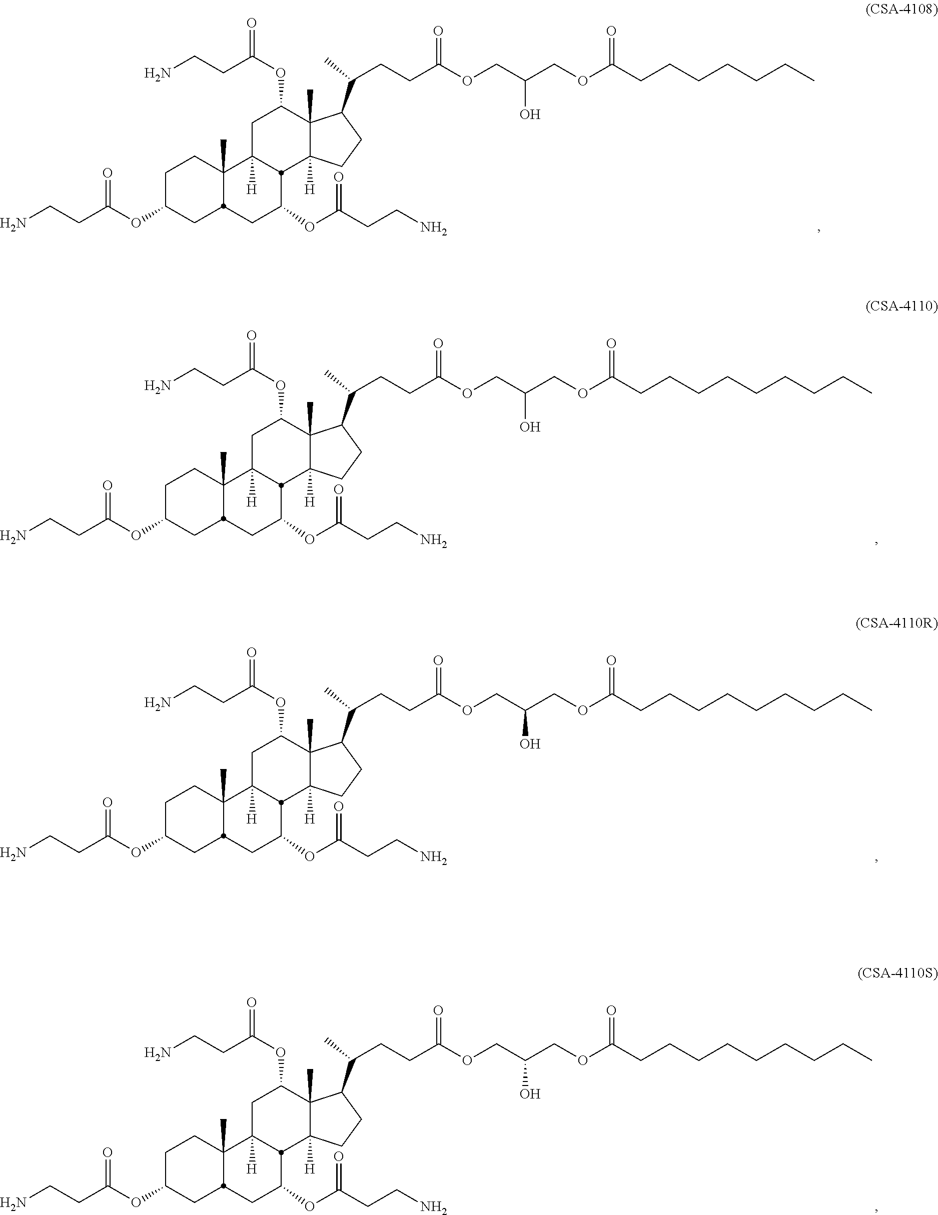

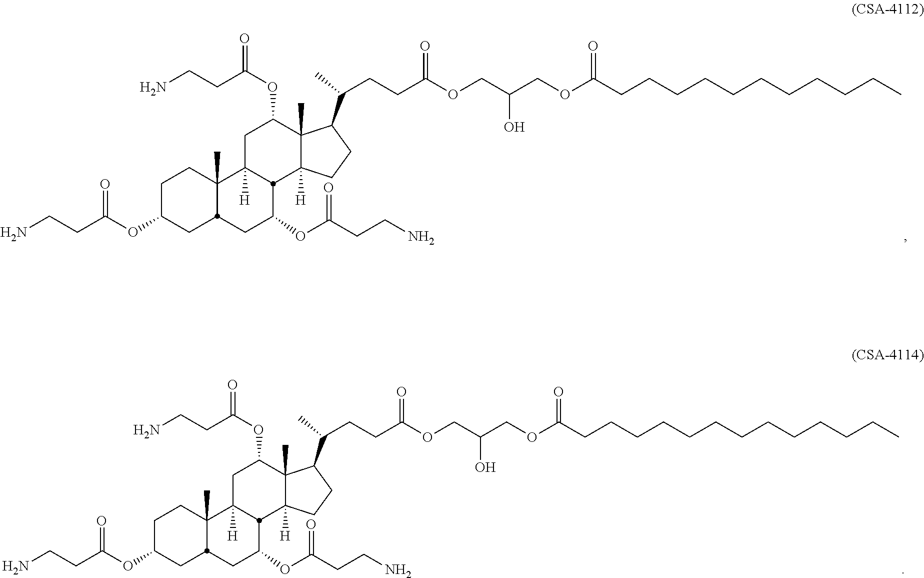

Non-limiting examples of bioresorbable diglyceride CSA compounds that form bioresorbable degradation products by hydrolysis of ester groups are CSA-4204, CSA-4206, CSA-4208, CSA-4210, and CSA-4310, and salts thereof:

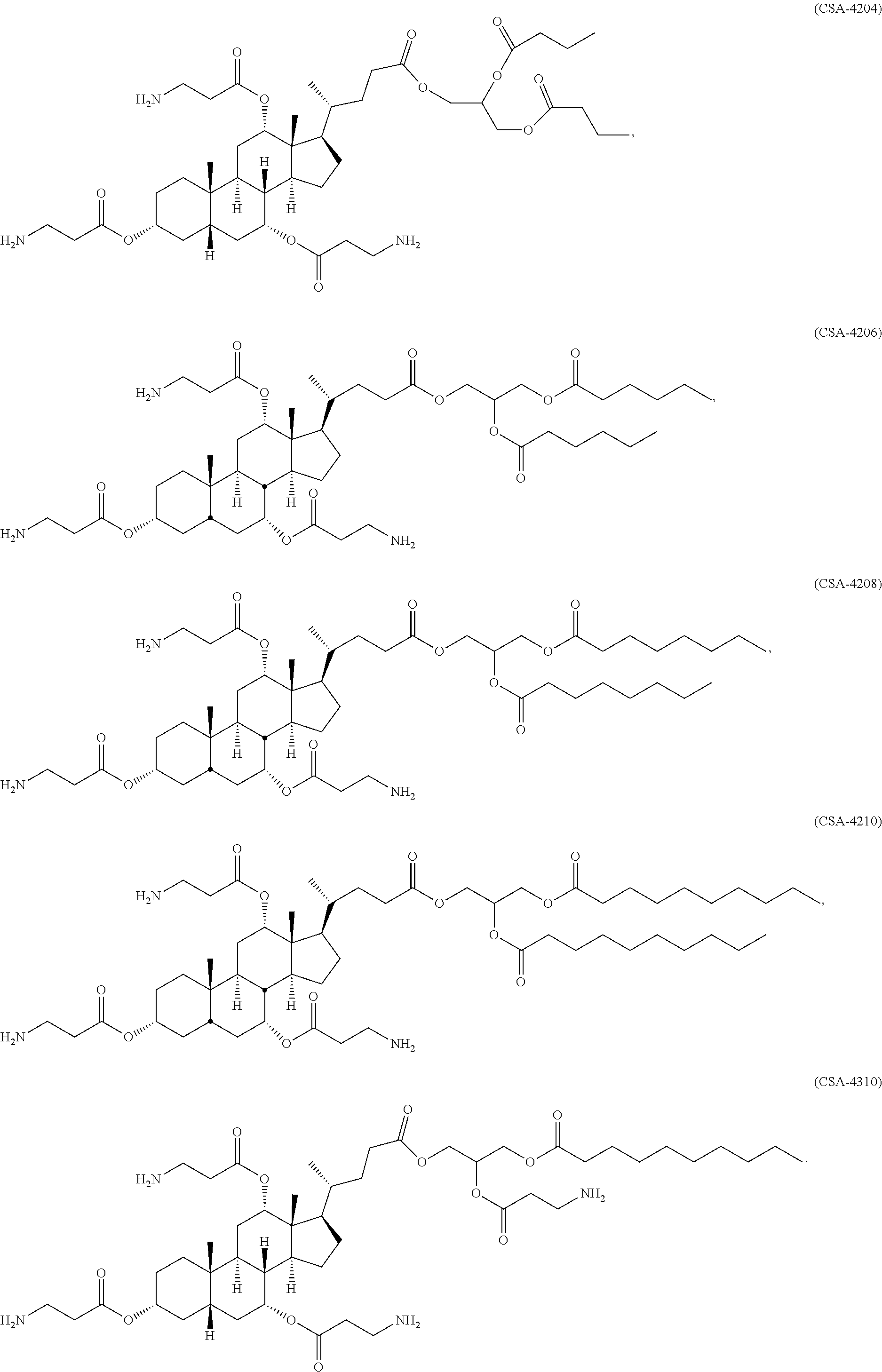

The degradation products of CSA-4108, CSA-4110, CSA-4110R, CSA-4110S, CSA-4112, CSA-4114, CSA-4204, CSA-4206, CSA-4208, CSA-4210, CSA-4310, and salts thereof. comprise a plurality of the following fully bioresorbable molecules:

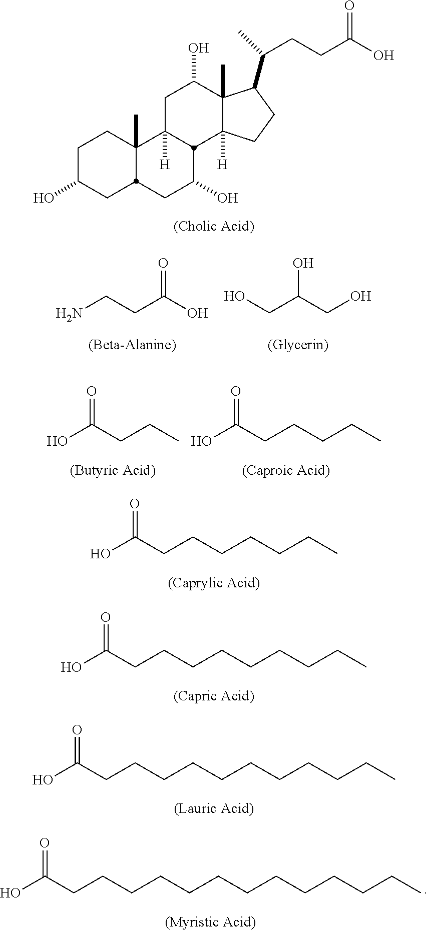

In some embodiments, a method of manufacturing a bioresorbable CSA compound with glyceride and other ester linkages includes: (1) reacting the C24 acid group of cholic acid with a protecting group (e.g., benzyl chloride) to yield a benzyl (Bn) ester of cholic acid; (2) reacting at least one of the hydroxyl groups at the C3, C7 and C12 positions of the protected cholic acid with an N-protected amino acid (e.g., tert-butyloxycarbonyl (BOC) protected beta-alanine) to form one or more ester linkages that link at least one of R₃, R₇ and R₁₂ to the sterol backbone at the corresponding C3, C7 and C12 position(s); (3) deprotecting the protected C24 acid group of cholic acid; (4) reacting the deprotected C24 acid group with (i) a monoglyceride of a fatty acid to form a glyceride linkage, (ii) a diglyceride of one or more fatty acids to form a glyceride linkage, (iii) or a mixed diglyceride of a fatty acid and an N-protected amino acid to form a glyceride linkage; and (5) deprotecting the amino group(s) of the one or more R₃, R₇ and R₁₂ amino acid ester groups in the case of (i), (ii), and (iii) and also the amino group of the R₁₈ in the case of (iii), to yield a desired bioresorbable CSA compound.

Advantages of bioresorbable CSA compounds disclosed herein include, but are not limited to, forming fully resorbable degradation products, such as cholic acid, amino acids (e.g., beta-alanine), glycerin, and fatty acids (e.g., C₂-C₂₂ fatty acids, preferably C₄-C₁₄ fatty acids), and providing comparable and/or improved antimicrobial activity, anti-inflammatory activity, and other desired properties compared to existing CSA compounds and/or simplified synthesis of CSA compounds and/or intermediate CSA compounds compared to existing synthetic routes.

Additional features and advantages will be set forth in part in the description that follows, and in part will be obvious from the description, or may be learned by practice of the embodiments disclosed herein. It is to be understood that both the foregoing brief summary and the following detailed description are exemplary and not restrictive of the embodiments disclosed herein or as claimed.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings, in which:

FIG. 1 is a graph that compares the minimum inhibitory concentrations (MIC) of various bioresorbable cationic steroidal antimicrobial (CSA) compounds of the invention;

FIG. 2 is a graph that compares the minimum bactericidal concentrations (MBC) and minimum fungicidal concentrations (MBC) of various bioresorbable CSA compounds of the invention;

FIGS. 3A-3D are graphs that compare the effects of pH and lipase on the hydrolysis rate of CSA-4110, a bioresorbable CSA compound of the invention, in aqueous solutions;

FIG. 4 is a graph that shows the effects of various concentrations of sodium dodecyl sulfate (SDS), CSA-44, CSA-13, and CSA-4110 on the absorbance of orange OT dye; and

FIG. 5 is a graph that shows the effects of CSA-131 or CSA-4110 on the fluorescence intensity of the dye 3,3′-diethylthiadicarbocyanine iodide (DiSC2 (5)) in the presence of MRSA ATCC BAA-42 (CSA-131 or CSA-4110 was added at 140 seconds to achieve 8 μg/mL after addition).

### DETAILED DESCRIPTION

Disclosed herein is a new class of cationic steroidal antimicrobial (CSA) compounds with bioresorbable groups attached to a bioresorbable sterol backbone via ester or other hydrolysable linkages, include mono- and diglyceride linkages. This class of CSA compounds is referred to as “bioresorbable CSA compounds”.

The degradation products of bioresorbable CSA compounds are themselves bioresorbable, such as cholic acid (a common bile acid), amino acids (e.g., beta-alanine, a bioresorbable neurotransmitter and amino acid), glycerin, and fatty acids, e.g., short-chain, medium-chain, and long-chain fatty acids. The bioresorbable CSA compounds are relatively easy and inexpensive to manufacture, are safe at higher concentrations, and possess desired antimicrobial, anti-inflammatory, and other desirable properties. Additionally, the bioresorbable CSA compounds retain activity against bacteria that are resistant to other antibiotics, including colistin.

### Definitions

“R” groups such as, without limitation, R₁, R₂, R₃, R₄, R₅, R₆, R₇, R₈, R₉, R₁₀, R₁₁, R₁₂, R₁₃, R₁₄, R₁₅, R₁₆, R₁₇, and R₁₈, represent substituents that can be attached to the sterol backbone. Unless otherwise specified, an R group may be substituted or unsubstituted.

A “ring” can be heterocyclic or carbocyclic. “Saturated” means a ring in which each atom is either hydrogenated or substituted such that the valency of each atom is filled. “Unsaturated” means a ring where the valency of each atom of the ring may not be filled with hydrogen or other substituents. For example, adjacent carbon atoms in a fused ring can be double bonded to each other. Unsaturation can also include deleting at least one of the following pairs and completing the valency of the ring carbon atoms at these deleted positions with a double bond, such as R₈ and R₉; R₈ and R₁₀; and R₁₃ and R₁₄.

Where a group is “substituted” it may be substituted with one, two, three or more of the indicated substituents, which may be the same or different, each replacing a hydrogen atom. If no substituents are indicated, the indicated “substituted” group may be substituted with one or more groups individually and independently selected from alkyl, alkenyl, alkynyl, cycloalkyl, cycloalkenyl, cycloalkynyl, acylalkyl, alkoxyalkyl, aminoalkyl, amino acid, aryl, heteroaryl, heteroalicyclyl, aralkyl, heteroaralkyl, (heteroalicyclyl) alkyl, hydroxy, protected hydroxyl, alkoxy, aryloxy, acyl, mercapto, alkylthio, arylthio, cyano, halogen (e.g., F, Cl, Br, and I), thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, oxo, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group, RₐO(CH₂)ₘO—, R_{b}(CH₂)ₙO—, R_{c}C(O)O(CH₂)ₚO—, and protected derivatives thereof. The substituent may be attached to the group at more than one attachment point. For example, an aryl group may be substituted with a heteroaryl group at two attachment points to form a fused multicyclic aromatic ring system. Biphenyl and naphthalene are two examples of an aryl group that is substituted with a second aryl group. A group that is not specifically labeled as substituted or unsubstituted may be considered to be either substituted or unsubstituted.

The terms “Cₐ” or “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having 1 to 4 carbons, that is, CH₃-, CH₃CH₂—, CH₃CH₂CH₂—, (CH₃)₂CH—, CH₃CH₂CH₂CH₂—, CH₃CH₂CH(CH₃)—, (CH₃)₂CHCH₂— and (CH₃)₃C—. If no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

“Alkyl” refers to a straight or branched hydrocarbon chain that comprises a fully saturated (no double or triple bonds) hydrocarbon group. The alkyl group may have 1 to 25 carbon atoms (whenever it appears herein, a numerical range such as “1 to 25” refers to each integer in the given range; e.g., “1 to 25 carbon atoms” means that the alkyl group may consist of 1 carbon atom, 2 carbon atoms, 3 carbon atoms, etc., up to and including 25 carbon atoms, although the present definition also covers the occurrence of the term “alkyl” where no numerical range is designated). The alkyl group may also be a medium size alkyl having 1 to 15 carbon atoms. The alkyl group could also be a lower alkyl having 1 to 6 carbon atoms. The alkyl group of the compounds may be designated as “C_{4”} or “C₁-C₄ alkyl” or similar designations. By way of example, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

“Alkenyl” refers to an alkyl group that contains in the straight or branched hydrocarbon chain one or more double bonds. The alkenyl group may have 2 to 25 carbon atoms (whenever it appears herein, a numerical range such as “2 to 25” refers to each integer in the given range; e.g., “2 to 25 carbon atoms” means that the alkenyl group may consist of 2, 3, or 4 carbon atoms, etc., up to and including 25 carbon atoms, although the present definition also covers the occurrence of the term “alkenyl” where no numerical range is designated). The alkenyl group may also be a medium size alkenyl having 2 to 15 carbon atoms. The alkenyl group could also be a lower alkenyl having 1 to 6 carbon atoms. The alkenyl group of the compounds may be designated as “C₄” or “C₂-C₄ alkenyl” or similar designations. An alkenyl group may be unsubstituted or substituted.

“Alkynyl” refers to an alkyl group that contains in the straight or branched hydrocarbon chain one or more triple bonds. The alkynyl group may have 2 to 25 carbon atoms (whenever it appears herein, a numerical range such as “2 to 25” refers to each integer in the given range; e.g., “2 to 25 carbon atoms” means that the alkynyl group may consist of 2, 3, or 4 carbon atoms, etc., up to and including 25 carbon atoms, although the present definition also covers the occurrence of the term “alkynyl” where no numerical range is designated). The alkynyl group may also be a medium size alkynyl having 2 to 15 carbon atoms. The alkynyl group could also be a lower alkynyl having 2 to 6 carbon atoms. The alkynyl group of the compounds may be designated as “C₄” or “C₂-C₄ alkynyl” or similar designations. An alkynyl group may be unsubstituted or substituted.

“Aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group (although the definition of C₆-C₁₀ aryl covers the occurrence of “aryl” when no numerical range is designated). Examples of aryl groups include, but are not limited to, benzene, naphthalene and azulene. An aryl group may be substituted or unsubstituted.

“Aralkyl” and “aryl(alkyl)” refer to an aryl group connected, as a substituent, via a lower alkylene group. The aralkyl group may have 6 to 20 carbon atoms (whenever it appears herein, a numerical range such as “6 to 20” refers to each integer in the given range; e.g., “6 to 20 carbon atoms” means that the aralkyl group may consist of 6 carbon atom, 7 carbon atoms, 8 carbon atoms, etc., up to and including 20 carbon atoms, although the present definition also covers the occurrence of the term “aralkyl” where no numerical range is designated). The lower alkylene and aryl group of an aralkyl may be substituted or unsubstituted. Examples include but are not limited to benzyl, 2-phenylalkyl, 3-phenylalkyl, and naphthylalkyl.

“Lower alkylene groups” refers to a C₁-C₂₅ straight-chained alkyl tethering groups, such as —CH₂— tethering groups, forming bonds to connect molecular fragments via their terminal carbon atoms. Examples include but are not limited to methylene (—CH₂—), ethylene (—CH₂CH₂—), propylene (CH₂CH₂CH₂—), and butylene (CH₂CH₂CH₂CH₂—). A lower alkylene group can be substituted by replacing one or more hydrogen of the lower alkylene group with a substituent(s) listed under the definition of “substituted.”

“Cycloalkyl” refers to a completely saturated (no double or triple bonds) mono-or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

“Cycloalkenyl” refers to a mono-or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl group may be unsubstituted or substituted.

“Cycloalkynyl” refers to a mono-or multi-cyclic hydrocarbon ring system that contains one or more triple bonds in at least one ring. If there is more than one triple bond, the triple bonds cannot form a fully delocalized pi-electron system throughout all the rings. When composed of two or more rings, the rings may be joined together in a fused fashion. A cycloalkynyl group may be unsubstituted or substituted.

“Alkoxy” or “alkyloxy” refer to the formula —OR wherein R is an alkyl, an alkenyl, an alkynyl, a cycloalkyl, a cycloalkenyl or a cycloalkynyl as defined above. A non-limiting list of alkoxys are methoxy, ethoxy, n-propoxy, 1-methylethoxy (isopropoxy), n-butoxy, iso-butoxy, sec-butoxy and tert-butoxy. An alkoxy may be substituted or unsubstituted.

“Acyl” refers to a hydrogen, alkyl, alkenyl, alkynyl, aryl, or heteroaryl connected, as substituents, via a carbonyl group, such as —(C═O)-R. Examples include formyl, acetyl, propanoyl, benzoyl, and acryl. An acyl may be substituted or unsubstituted.

“Alkoxyalkyl” or “alkyloxyalkyl” refer to an alkoxy group connected, as a substituent, via a lower alkylene group. Examples include alkyl-O-alkyl- and alkoxy-alkyl-with the terms alkyl and alkoxy defined herein.

“Hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

“Haloalkyl” refers to an alkyl group in which one or more of the hydrogen atoms are replaced by a halogen (e.g., mono-haloalkyl, di-haloalkyl and tri-haloalkyl). Examples include chloromethyl, fluoromethyl, difluoromethyl, trifluoromethyl and 1-chloro-2-fluoromethyl, 2-fluoroisobutyl. A haloalkyl may be substituted or unsubstituted.

“Amino” refers to “—NH₂”.

“Hydroxy” refers to “—OH”.

“Cyano” refers to “—CN”.

“Carbonyl” or “oxo” refer to “—C═O”.

“Azido” refers to “—N₃”.

“Aminoalkyl” refers to an amino group connected, as a substituent, via a lower alkylene group. Examples include H₂N-alkyl- with the term alkyl defined herein.

“Alkylcarboxyalkyl” refers to an alkyl group connected, as a substituent, to a carboxy group that is connected, as a substituent, to an alkyl group. Examples include alkyl-(C═O)—O—alkyl- and alkyl-O-(C═O)-alkyl- with the term alkyl as defined herein.

“Alkylaminoalkyl” refers to an alkyl group connected, as a substituent, to an amino group that is connected, as a substituent, to an alkyl group. Examples include alkyl-NH-alkyl- with the term alkyl as defined herein.

“Dialkylaminoalkyl” and “di(alkyl)aminoalkyl” refer to two alkyl groups connected,

each as a substituent, to an amino group that is connected, as a substituent, to an alkyl group. Examples include

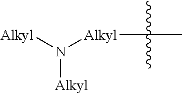

with the term alkyl as defined herein.

“Alkylaminoalkylamino” refers to an alkyl group connected, as a substituent, to an amino group that is connected, as a substituent, to an alkyl group that is connected, as a substituent, to an amino group. Examples include alkyl-NH-alkyl-NH— with the term alkyl as defined herein.

“Alkylaminoalkylaminoalkylamino” refers to an alkyl group connected, as a substituent, to an amino group that is connected, as a substituent, to an alkyl group that is connected, as a substituent, to an amino group that is connected, as a substituent, to an alkyl group. Examples include alkyl-NH-alkyl-NH-alkyl- with the term alkyl as defined herein.

“Arylaminoalkyl” refers to an aryl group connected, as a substituent, to an amino group that is connected, as a substituent, to an alkyl group. Examples include aryl-NH-alkyl- with the terms aryl and alkyl as defined herein.

“Aminoalkyloxy” refers to an amino group connected, as a substituent, to an alkyloxy group. Examples include H₂N-alkyl-O— and H₂N-alkoxy- with the terms alkyl and alkoxy as defined herein.

“Aminoalkyloxyalkyl” refers to an amino group connected, as a substituent, to an alkyloxy group connected, as a substituent, to an alkyl group. Examples include H₂N-alkyl-O-alkyl- and H₂N-alkoxy-alkyl- with the terms alkyl and alkoxy as defined herein.

“Aminoalkylcarboxy” refers to an amino group connected, as a substituent, to an alkyl group connected, as a substituent, to a carboxy group. Examples include H₂N-alkyl-(C═O)—O—and H₂N-alkyl-O—(C═O)— with the term alkyl as defined herein.

“Aminoalkylaminocarbonyl” refers to an amino group connected, as a substituent, to an alkyl group connected, as a substituent, to an amino group connected, as a substituent, to a carbonyl group. Examples include H₂N-alkyl-NH—(C═O)— with the term alkyl as defined herein.

“Aminoalkylcarboxamido” refers to an amino group connected, as a substituent, to an alkyl group connected, as a substituent, to a carbonyl group connected, as a substituent to an amino group. Examples include H₂N-alkyl-(C═O)—NH— and H₂N-alkyl-NH—(C═O)— with the term alkyl as defined herein.

“Azidoalkyloxy” refers to an azido group connected as a substituent, to an alkyloxy group. Examples include N₃-alkyl-O— and N₃-alkoxy- with the terms alkyl and alkoxy as defined herein.

“Cyanoalkyloxy” refers to a cyano group connected as a substituent, to an alkyloxy group. Examples include NC-alkyl-O— and NC-alkoxy- with the terms alkyl and alkoxy as defined herein.

“Sulfenyl” refers to “—SR” in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl) alkyl. A sulfenyl may be substituted or unsubstituted.

“Sulfinyl” refers to “—(S═O)—R” in which R can be the same as defined with respect to sulfenyl. A sulfinyl may be substituted or unsubstituted.

“Sulfonyl” refers to “—(S═O)—OR” in which R can be the same as defined with respect to sulfenyl. A sulfonyl may be substituted or unsubstituted.

“O-carboxy” refers to “R-(C═O)—O—” in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl) alkyl, as defined herein. An O-carboxy may be substituted or unsubstituted.

“Ester” and “C-carboxy” refer to “—(C═O)—OR” in which R can be the same as defined with respect to O-carboxy. An ester and C-carboxy may be substituted or unsubstituted.

“Thiocarbonyl” refers to “—(C═S)—R” in which R can be the same as defined with respect to O-carboxy. A thiocarbonyl may be substituted or unsubstituted.

“Trihalomethanesulfonyl” refers to “X₃CSO₂—” wherein X is a halogen.

“S-sulfonamido” refers to “—SO₂N(RARB)” in which RA and RB can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl) alkyl. An S-sulfonamido may be substituted or unsubstituted.

“N-sulfonamido” refers to “RSO₂N(RA)-” in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl) alkyl. An N-sulfonamido may be substituted or unsubstituted.

“O-carbamyl” and “urethanyl” refer to “—O—(C═O)—N(RARB)” in which RA and RB can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl) alkyl. An O-carbamyl or urethanyl may be substituted or unsubstituted.

“N-carbamyl” refers to “RO—(C═O)—N(RA)-” in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl) alkyl. An N-carbamyl may be substituted or unsubstituted.

“O-thiocarbamyl” refers to “—O—(C═S)—N(RARB)” in which RA and RB can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl) alkyl. An O-thiocarbamyl may be substituted or unsubstituted.

“N-thiocarbamyl” refers to “RO—(C═S)—N(RA)-” in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl) alkyl. An N-thiocarbamyl may be substituted or unsubstituted.

C-amido” refers to “—(C═O)—N(RARB)” in which RA and RB can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl) alkyl. A C-amido may be substituted or unsubstituted.

“N-amido” refers to “R—(C═O)—N(RA)-” in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl) alkyl. An N-amido may be substituted or unsubstituted.

“Guanidinoalkyloxy” refers to a guanidinyl group connected, as a substituent, to an alkyloxy group. Examples include

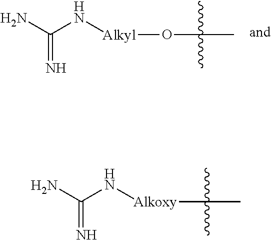

with the terms alkyl and alkoxy as defined herein.

“Guanidinoalkylcarboxy” refers to a guanidinyl group connected, as a substituent, to an alkyl group connected, as a substituent, to a carboxy group. Examples include

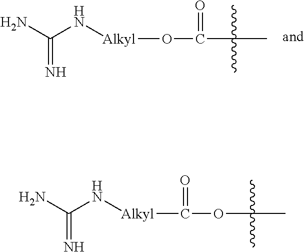

with the term alkyl as defined herein.

“Quaternary ammonium alkylcarboxy” refers to a quaternary amino group connected, as a substituent, to an alkyl group connected, as a substituent, to a carboxy group. Examples include

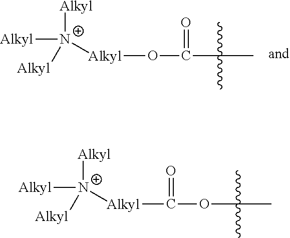

with the term alkyl as defined herein.

“Halogen atom” and “halogen” mean any one of the radio-stable atoms of column 7 of the Periodic Table of the Elements, such as, fluorine, chlorine, bromine, and iodine.

Where the number of substituents is not specified (e.g., haloalkyl), there may be one or more substituents present. For example, “haloalkyl” may include one or more of the same or different halogens.

“Amino acid” refers to any amino acid (both standard and non-standard amino acids), including, but not limited to, α-amino acids, β-amino acids, γ-amino acids and δ-amino acids. Examples of suitable amino acids include, but are not limited to, alanine, asparagine, aspartate, cysteine, glutamate, glutamine, glycine, proline, serine, tyrosine, histidine, isoleucine, leucine, lysine, methionine, phenylalanine, threonine, tryptophan, and valine. Additional examples of suitable amino acids include, but are not limited to, ornithine, arginine, hypusine, 2-aminoisobutyric acid, dehydroalanine, γ-aminobutyric acid, citrulline, β-alanine, α-ethyl-glycine, α-propyl-glycine, and norleucine.

A linking group is a divalent moiety used to link one CSA compound to another. In embodiments, the linking group is used to link a first CSA with a second CSA (which may be the same or different). An example of a linking group is (C₁-C₁₀) alkyloxy-(C₁-C₁₀) alkyl.

“P.G.” or “protecting group” or “protecting groups” refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl (Bn); substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl)ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein). Amino-protecting groups are known to those skilled in the art. In general, the species of protecting group is not critical, provided that it is stable to the conditions of any subsequent reaction(s) on other positions of the compound and can be removed at the appropriate point without adversely affecting the remainder of the molecule. In addition, a protecting group may be substituted for another after substantive synthetic transformations are complete. Clearly, where a compound differs from a compound disclosed herein only in that one or more protecting groups of the disclosed compound has been substituted with a different protecting group, that compound is within the disclosure.

### CSA Compounds

Cationic steroidal antimicrobial (CSA) compounds, also referred to as “CSA compounds”, “CSAs”, “CSA molecules”, “ceragenins” or “ceragenin compounds”, are synthetically produced, small molecule chemical compounds that include a sterol backbone having various charged groups (e.g., amine and cationic groups) attached to the backbone. The sterol backbone can be used to orient amine or guanidine groups on a face or plane of the sterol backbone. CSAs are cationic and amphiphilic, based upon the functional groups attached to the backbone. They are facially amphiphilic with a hydrophobic face and a polycationic face.

Without wishing to be bound to theory, CSA compounds described herein act as anti-microbial agents (e.g., anti-bacterial, anti-fungal, and anti-viral). It is believed, for example, that anti-microbial CSA compounds may act as an antimicrobial by binding to the cellular membrane of bacteria and other microbes and modifying the cell membrane, e.g., such as by forming a pore that allows the leakage of ions and cytoplasmic materials critical to the microbe's survival, and leading to the death of the affected microbe. In addition, anti-microbial CSA compounds may also act to sensitize bacteria to other antibiotics. For example, at concentrations of anti-microbial CSA compounds below the corresponding minimum bacteriostatic concentration (MIC), the CSA compound may cause bacteria to become more susceptible to other antibiotics by disrupting the cell membrane, such as by increasing membrane permeability. It is postulated that charged cationic groups may be responsible for disrupting the bacterial cellular membrane and imparting anti-microbial properties. CSA compounds may have similar membrane-or outer coating-disrupting effects on fungi and viruses.

By way of background, exemplary CSA compounds and methods for their manufacture are described in U.S. Pat. Nos. 6,350,738, 6,486,148, 6,767,904, 7,598,234, 7,754,705, 8,691,252, 8,975,310, 9,434,759, 9,527,883, 9,943,614, 10,155,788, 10,227,376, 10,370,403, 10,626,139, 11,286,276, and 12,030,912, and U.S. Pat. Pub. Nos. 2016/0311850 and 2021/0363174, which are incorporated herein by reference. The skilled artisan will recognize the compounds within the generic formulae set forth herein and understand their preparation in view of the references cited herein and the Examples.

The CSA compounds and compositions disclosed herein are optionally prepared as salts, which advantageously makes them cationic when one or more amine groups is/are protonated. The term “salt” as used herein is a broad term, is to be given its ordinary and customary meaning to a skilled artisan (and is not to be limited to a special or customized meaning) and refers without limitation to a salt of a compound. In embodiments, the salt is an acid addition salt of the compound. Salts can be obtained by reacting a compound with inorganic acids such as hydrohalic acid (e.g., hydrochloric acid or hydrobromic acid), sulfuric acid, nitric acid, phosphoric acid, and phosphonic acid. Salts can also be obtained by reacting a compound with an organic acid such as aliphatic or aromatic carboxylic or sulfonic acids, sulfinic acids, for example formic acid, acetic acid, propionic acid, glycolic acid, pyruvic acid, malonic acid, maleic acid, fumaric acid, trifluoroacetic acid, benzoic acid, cinnamic acid, mandelic acid, succinic acid, lactic acid, malic acid, tartaric acid, citric acid, ascorbic acid, nicotinic acid, methanesulfonic acid, ethanesulfonic acid, p-toluensulfonic acid, salicylic acid, stearic acid, muconic acid, butyric acid, phenylacetic acid, phenylbutyric acid, valproic acid, 1,2-ethanedisulfonic acid, 2-hydroxyethanesulfonic acid, benzenesulfonic acid, 2-naphthalenesulfonic acid, or 1,5-naphthalenedisulfonic acid (NDSA). Salts can also be obtained by reacting a compound with a base to form a salt such as an ammonium salt, an alkali metal salt, such as a lithium, sodium or a potassium salt, an alkaline earth metal salt, such as a calcium, magnesium or aluminum salt, a salt of organic bases such as dicyclohexylamine, N-methyl-D-glucamine, tris (hydroxymethyl) methylamine, C₁-C₇ alkylamine, cyclohexylamine, dicyclo-hexylamine, triethanolamine, ethylenediamine, ethanolamine, diethanolamine, triethanol-amine, tromethamine, and salts with amino acids such as arginine and lysine; or a salt of an inorganic base, such as aluminum hydroxide, calcium hydroxide, potassium hydroxide, sodium carbonate, sodium hydroxide, or the like.

In some embodiments, the salt is a hydrochloride salt. In embodiments, the salt is a mono-hydrochloride salt, a di-hydrochloride salt, a tri-hydrochloride salt, or a tetra-hydrochloride salt. Additional examples of salts include sulfuric acid addition salts, sulfonic acid addition salts, disulfonic acid addition salts, 1,5-naphthalenedisulfonic acid addition salts (e.g., di-addition salts), sulfate salts, and bisulfate salts.

The CSA compounds disclosed herein can have a structure of Formula I, II or III, or a salt thereof, having a steroidal backbone, and wherein at least one of R₁-R₁₈, preferably R₁₈, can include a mono-or diglyceride fatty acid ester group at the C24 position of the steroidal backbone, and at least one of R₁-R₁₈, preferably at least one of R₃, R₇ and R₁₂, can comprise an amino acid linked to the sterol backbone by an ester linkage at the C3, C7 and/or C12 positions(s):

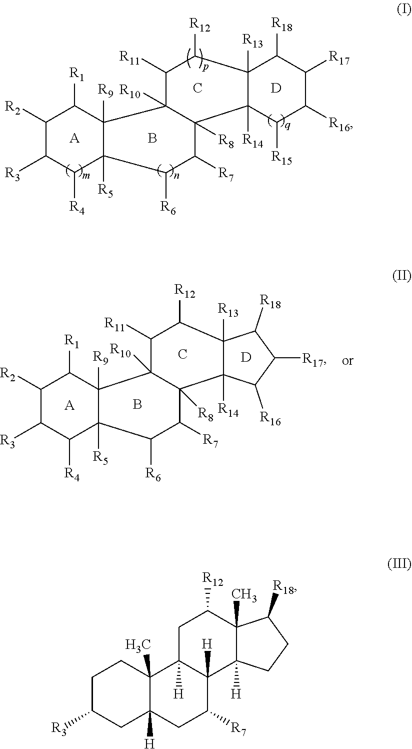

where R₁₅ is omitted in Formulas II and III.

In some embodiments, at least one of R₁-R₁₈, preferably R₁₈, can have the following mono-or diglyceride structure:

—R₁₉-(C═O)—O—C—CR₂₀—CR₂₁

where R₁₉ is omitted or is selected from alkyl, alkenyl, alkynyl, and aryl, and R₂₀ and R₂₁ are independently selected from hydroxy and (C₂-C₂₂)alkylcarboxy, provided that at least one of R₂₀ or R₂₁ is (C₂-C₂₂)alkylcarboxy, the (C₂-C₂₂)alkylcarboxy preferably having an even number of carbons. R₁₈ can be degraded (e.g., by hydrolysis of the glyceride ester groups at or that extend from the C24 position). Examples of (C2-C22)alkylcarboxy groups with an even number of carbons are acetic acid, butyric acid, caproic (hexanoic) acid, caprylic (octanoic) acid, capric (decanoic) acid, lauric (dodecanoic) acid, myristic (tetradecanoic) acid, palmitic (hexadecenoic) acid, stearic (octadecanoic) acid, arachidic (icosanoic) acid, and behenic (docosanoic) acid. Non-limiting examples of (C₂-C₂₂)alkylcarboxy groups with an odd number of carbons that are bioresorbable are propionic acid and valeric (pentanoic) acid.

It is within the scope of the invention to use other carboxylate groups that form natural carboxylic acids as degradation products, such as isomers, analogs, and derivatives of (C₂-C₂₂)alkylcarboxy groups. For example, it is within the scope of the invention to form glycerides from unsaturated fatty acids, such as monounsaturated fatty acids and/or polyunsaturated fatty acids, such as oleic acid, omega-3 fatty acids, omega-6 fatty acids, and omega-9 fatty acids. In some embodiments, R₁₈ can have the following mixed diglyceride structure:

—R₁₉—(C═O)—O—C—CR₂₀—CR₂₁

where R₁₉ is omitted or is selected from alkyl, alkenyl, alkynyl, and aryl, R₂₀ is a (C₂-C₂₂)alkylcarboxy, the (C₂-C₂₂)alkylcarboxy preferably having an even number of carbons, and R₂₁ can have the following aminoalkylcarboxy structure:

R₂₄R₂₃N—R₂₂—(C═O)—O—

where R₂₂ is a substituted or unsubstituted alkyl and R₂₃ and R₂₄ are independently selected from hydrogen, alkyl, alkenyl, alkynyl, and aryl. R₂₂ is preferably an ester group of an amino acid, such as beta-alanine, which forms a bioresorbable amino acid (e.g., beta-alanine) as degradation product (e.g., by hydrolysis of the ester groups at the C24). The glyceride portion forms bioresorbable glycerin, a fatty acid, and an amino acid as degradation products (e.g., by hydrolysis of ester groups in the glyceride structure).

In some embodiments, at least one of R₁-R₁₈, preferably at least one, more preferably at least two, and even more preferably three, of R₃, R₇ and R₁₂, can have the following aminoalkylcarboxy or aminoalkylcarboxamido structure:

R₂₄R₂₃N—R₂₂—(C═O)—X—

where R₂₂ is a substituted or unsubstituted alkyl, X is oxygen or nitrogen, and R₂₃ and R₂₄ are independently selected from hydrogen, alkyl, alkenyl, alkynyl, and aryl. At least one of R₃, R₇ and R₁₂, preferably two or three of R₃, R₇ and R₁₂, is/are an ester group of one or more amino acids, such as beta-alanine, which forms a bioresorbable amino acid (e.g., beta-alanine) as degradation product (e.g., by hydrolysis of the ester group(s) at the C3, C7 and/or C12position(s)). Alternatively, the aminoalkyl portion of at least one of R₃, R₇ and R₁₂ can be attached to one or more of the C3, C7 and/or C12 positions of the sterol backbone (or elsewhere) by other linkages, such as amide or ether linkage.

It is within the scope of the invention to use other amino acid derivatives that form other naturally occurring amino acids as degradation products, such as D-alanine, L-alanine, L-asparagine, D-aspartic acid, L-aspartic acid, L-cysteine, L-glutamic acid, L-glutamine, glycine, L-proline, D-serine, L-serine, L-tyrosine, L-histidine, L-isoleucine, L-leucine, L-lysine, D-methionine, L-methionine, L-phenylalanine, L-threonine, L-tryptophan, D-valine, L-valine, L-ornithine, L-arginine, hypusine, 2-aminoisobutyric acid, dehydroalanine, γ-aminobutyric acid, L-citrulline, α-ethyl-glycine, α-propyl-glycine, and L-norleucine.

The aminoalkyl group of at least one, such as one or two, of R₃, R₇, and R₁₂ can be attached to the sterol backbone by other linkages, such as amide or ether linkage, such as where

R₃, R₇, and/or R₁₂ has one of the following alternative structures:

R₂₄R₂₃N—R₂₂—(C═O)—N— (amide)

R₂₅R₂₃N—R₂₂—O— (ether)

where R₂₂, R₂₃ and R₂₄ are as defined above. Such linkages are not as easily hydrolyzed as ester linkages.

Referring back to Formulae I, II and III above, when the CSA compound has a structure of Formula I, m, n, p, and q are independently 0 or 1.

When the CSA compound has a structure of Formula I or II,
rings A, B, C, and D are independently saturated, or are fully or partially unsaturated, provided that at least two of rings A, B, C, and D are saturated,
R₁ through R₁₈ are independently selected from the group consisting of hydrogen, hydroxyl, alkyl, hydroxyalkyl, alkyloxyalkyl, alkylcarboxyalkyl, alkylaminoalkyl, alkylaminoalkylamino, alkylaminoalkylaminoalkylamino, aminoalkyl, aryl, arylaminoalkyl, haloalkyl, alkenyl, alkynyl, oxo, linking group attached to a second steroid, aminoalkylurethanyl, aminoalkenylurethanyl, aminoalkynylurethanyl, aminoarylurethanyl, aminoalkyloxy, aminoalkylcarboxy, aminoalkyloxyalkyl, aminoalkylaminocarbonyl, aminoalkylcarboxamido, di (alkyl) aminoalkyl, H₂N—HC (Q₅)—(C═O)—O—, H₂N-H (Q₅)-(C═O)—NH—, azidoalkyloxy, cyanoalkyloxy, P.G.-HN—HC (Q₅)—(C═O)—O—, guanidino-alkyloxy, quaternary ammonium alkylcarboxy, and guanidinoalkyl carboxy, where Q₅ is a side chain of any amino acid (including a side chain of glycine, i.e., H), and P.G. is an amino protecting group; and
R₅, R₈, R₉, R₁₀, R₁₃, R₁₄ and R₁₇ are independently deleted when one of rings A, B, C, or D is unsaturated so as to complete the valency of the carbon atom at that site,
provided that at least one of Ri through R₄, R₆, R₇, R₁₁, R₁₂, R₁₅, R₁₆, and R₁₈, preferably R₁₈, includes a glyceride linkage with one or more fatty acid ester groups, such butyl, hexyl, octyl, decyl, dodecyl, or tetradecyl ester groups, attached to the sterol backbone, e.g., at the C24 position, by a glyceride ester linkage, and
provided that at least one of Ri through R₄, R₆, R₇, R₁₁, R₁₂, R₁₅, R₁₆, and R₁₈, preferably one, two, or three of R₃, R₇ and R₁₂, include(s) an amino acid attached to the sterol backbone, such as at the C3, C7 and/or C12 positions, by an ester linkage or amide linkage.

In some embodiments, R₁ through R₁₈ are independently selected from the group consisting of hydrogen, hydroxyl, substituted or unsubstituted (C₁-C₂₂)alkyl, substituted or unsubstituted (C₁-C₂₂) hydroxyalkyl, substituted or unsubstituted (C₁-C₂₂)alkyloxy-(C₁-C₂₂)alkyl, substituted or unsubstituted (C₁-C₂₂)alkylcarboxy-(C₁-C₂₂)alkyl, substituted or unsubstituted (C₁-C₂₂)alkylamino-(C₁-C₂₂)alkyl, substituted or unsubstituted (C₁-C₂₂)alkylamino-(C₁-C₂₂)alkylamino, substituted or unsubstituted (C₁-C₂₂)alkylamino-(C₁-C₂₂)alkylamino-(C₁-C₂₂)alkylamino, substituted or unsubstituted (C₁-C₂₂)aminoalkyl, substituted or unsubstituted aryl, substituted or unsubstituted arylamino-(C₁-C₂₂)alkyl, substituted or unsubstituted (C₁-C₂₂)haloalkyl, substituted or unsubstituted (C₂-C₆)alkenyl, substituted or unsubstituted (C₂-C₆)alkynyl, oxo, linking group attached to a second steroid, substituted or unsubstituted (C₁-C₂₂)aminoalkylurethanyl, substituted or unsubstituted (C₂-C₂₂) aminoalkenylurethanyl, substituted or unsubstituted (C₂-C₂₂) aminoalkynylurethanyl, substituted or unsubstituted aminoarylurethanyl, substituted or unsubstituted (C₁-C₂₂)aminoalkyloxy, substituted or unsubstituted (C₁-C₂₂)aminoalkylcarboxy, substituted or unsubstituted (C₁-C₂₂)aminoalkyloxy-(C₁-C₂₂)alkyl, substituted or unsubstituted (C₁-C₂₂)aminoalkyl-aminocarbonyl, substituted or unsubstituted (C₁-C₂₂)aminoalkylcarboxamido, substituted or unsubstituted di (C₁-C₂₂)alkylamino-(C₁-C₂₂)alkyl, H₂N—HC(Q₅)-(C═O)—O—, H₂N—HC(Q₅)-(C═O)—NH—, substituted or unsubstituted (C₁-C₂₂)azidoalkyloxy, substituted or unsubstituted (C₁-C₂₂) cyanoalkyloxy, P.G.-HN—HC (Q₅)-(C═O)—O—, substituted or unsubstituted (C₁-C₂₂) guanidinoalkyloxy, substituted or unsubstituted quaternary ammonium (C₁-C₂₂)alkylcarboxy, and substituted or unsubstituted (C₁-C₂₂) guanidinoalkyl carboxy, where Q₅ is a side chain of an amino acid (including a side chain of glycine, i.e., H), and P.G. is an amino protecting group; and
R₅, R₈, R₉, R₁₀, R₁₃, R₁₄ and R₁₇ are independently deleted when one of rings A, B, C, or D is unsaturated so as to complete the valency of the carbon atom at that site,
provided that at least one of Ri through R₄, R₆, R₇, R₁₁, R₁₂, R₁₅, R₁₆, and R₁₈, preferably R₁₈, includes a mono-or diglyceride fatty acid ester, such as a C₂-C₂₂)alkylcarboxy ester, attached to the sterol backbone, e.g., at the C24 position, and
provided that at least one of Ri through R₄, R₆, R₇, R₁₁, R₁₂, R₁₅, R₁₆, and R₁₈, preferably one, two or three of R₃, R₇ and R₁₂, include(s) an amino acid attached to the sterol backbone, such as at the C3, C7 and/or C12 positions, by an ester linkage or amide linkage.

In some embodiments, R₁, R₂, R₄, R₅, R₆, R₈, R₉, R₁₀, R₁₁, R₁₃, R₁₄, R₁₅, R₁₆, and R₁₇ are independently selected from the group consisting of hydrogen and unsubstituted (C₁-C₆) alkyl.

In some embodiments, R₁, R₂, R₄, R₅, R₆, R₈, R₁₀, R₁₁, R₁₄, R₁₆, and R₁₇ are each hydrogen and R₉ and R₁₃ are each methyl.

In some embodiments, one or more of rings A, B, C, and D is/are heterocyclic.

In some embodiments, rings A, B, C, and D is/are non-heterocyclic.

In some embodiments, the CSA compound is a compound of Formula III, which is a subgenus of Formula I and Formula II with specified stereochemistry, wherein R₁, R₂, R₄, R₅, R₆, R₈, R₁₀, R₁₁, R₁₄, and R₁₆, are hydrogen or methyl as shown, and Ris is omitted:

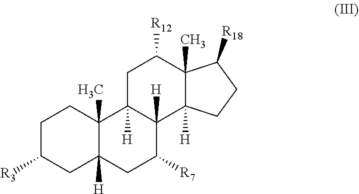

where R₃, R₇, R₁₂, and R₁₈ are defined as above for Formula I and II, such as where:

In some embodiments, R₁₈ has the following mono-or diglyceride structure:

-R₁₉-(C═O)—O—C—CR₂₀—CR₂₁

where R₁₉ is omitted or is selected from alkyl, alkenyl, alkynyl, and aryl, and R₂₀ and R₂₁ are independently selected from hydroxy and (C₂-C₂₂)alkylcarboxy, provided that at least one of R₂₀ or R₂₁ is (C₂-C₂₂)alkylcarboxy, the (C₂-C₂₂)alkylcarboxy preferably having an even number of carbons.

In some embodiments, R₁₈, has the following mixed diglyceride structure:

—R₁₉—(C═O)—O—C—CR₂₀—CR₂₁

where R₁₉ is omitted or is selected from alkyl, alkenyl, alkynyl, and aryl, R₂₀ is a (C₂-C₂₂)alkylcarboxy, the (C₂-C₂₂)alkylcarboxy preferably having an even number of carbons, and R₂₁ can have the following aminoalkylcarboxy structure:

R₂₄R₂₃N—R₂₂—(C═O)—O—

where R₂₂ is a substituted or unsubstituted alkyl and R₂₃ and R₂₄ are independently selected from hydrogen, alkyl, alkenyl, alkynyl, and aryl. R₂₂ is preferably an ester group of an amino acid, such as beta-alanine, which forms a bioresorbable amino acid (e.g., beta-alanine) as degradation product (e.g., by hydrolysis of the ester groups at the C24). The glyceride portion forms bioresorbable glycerin, a fatty acid, and an amino acid as degradation products (e.g., by hydrolysis of ester groups in the glyceride structure).

At least one, preferably two or three, of R₃, R₇ and R₁₂ has the following aminoalkylcarboxy structure:

R₂₄R₂₃N—R₂₂—(C═O)—O—

where R₂₂, R₂₃ and R₂₄ are as defined above for Formula I and II.

In embodiments, where one or two of R₃, R₇, and R₁₂ independently has an aminoalkylcarboxy structure as defined herein, one or two of R₃, R₇, and R₁₂ are independently selected from the group consisting of hydrogen, (C₁-C₂₂)alkyl, (C₁-C₂₂) hydroxyalkyl, (C₁-C₂₂)alkyloxy-(C₁-C₂₂)alkyl, (C₁-C₂₂)alkylcarboxy-(C₁-C₂₂)alkyl, (C₁-C₂₂)alkylamino-(C₁-C₂₂)alkyl, (C₁-C₂₂)alkylamino-(C₁-C₂₂)alkylamino, (C₁-C₂₂)alkylamino-(C₁-C₂₂)alkylamino-(C₁-C₁₈)alkylamino, (C₁-C₂₂)aminoalkyl, arylamino-(C₁-C₂₂)alkyl, (C₁-C₂₂)aminoalkyloxy, (C₁-C₂₂)aminoalkylcarboxy, (C₁-C₂₂)aminoalkyloxycarbonyl, (C₁-C₂₂)aminoalkyloxy-(C₁-C₂₂)alkyl, (C₁-C₂₂)aminoalkylaminocarbonyl, (C₁-C₂₂)aminoalkyl-carboxamido, di (C₁-C₂₂)alkylaminoalkyl, (C₁-C₂₂) guanidinoalkyloxy, quaternary ammonium (C₁-C₂₂)alkylcarboxy, and (C₁-C₂₂) guanidinoalkyl carboxy.

Preferably, where one or two of R₃, R₇, and R₁₂ independently has an aminoalkylcarboxy structure as defined herein, one or two of R₃, R₇, and R₁₂ are independently selected from the group consisting of hydrogen, (C₁-C₆)alkyl, (C₁-C₆) hydroxyalkyl, (C₁-C₁₆)alkyloxy-(C₁-C₅)alkyl, (C₁-C₁₆)alkylcarboxy-(C₁-C₅)alkyl, (C₁-C₁₆)alkylamino-(C₁-C₅)alkyl, (C₁-C₁₆)alkylamino-(C₁-C₅)alkylamino, (C₁-C₁₆)alkylamino-(C₁-C₁₆)alkylamino-(C₁-C₅)alkylamino, (C₁-C₁₆)aminoalkyl, arylamino-(C₁-C₅)alkyl, (C₁-C₅)aminoalkyloxy, (C₁-C₁₆)aminoalkyloxy-(C₁-C₅)alkyl, (C₁-C₅)aminoalkylcarboxy, (C₁-C₅)aminoalkyloxycarbonyl, (C₁-C₅)aminoalkylaminocarbonyl, (C₁-C₅)aminoalkylcarbox-amido, di (C₁-C₅)alkylamino-(C₁-C₅) alkyl, (C₁-C₅) guanidinoalkyloxy, quaternary ammonium (C₁-C₁₆)alkylcarboxy, and (C₁-C₁₆) guanidinoalkylcarboxy.

In some embodiments, R₃, R₇, and R₁₂ are the same aminoalkylcarboxy group.

In some embodiments, R₃, R₇, and R₁₂ are the same aminoalkylcarboxamido group.

In some embodiments, one or two of R₃, R₇, and R₁₂ are aminoalkylcarboxy.

In some embodiments, one or two of R₃, R₇, and R₁₂ are aminoalkylcarboxamido.

In some embodiments, one or two of R₃, R₇, and R₁₂ are aminoalkyloxy.

In some embodiments, bioresorbable mono- and diglyceride CSA compounds can have a chiral center, such as in the glyceryl moiety, so as to form enantiomers that can be isolated rather than forming a racemic mixture. Unless otherwise specified, the examples of CSA compounds disclosed herein can be non-chiral, R- and S-enantiomers forming a racemic mixture, the R-enantiomer, or the S-enantiomer.

Non-limiting examples of bioresorbable monoglyceride CSA compounds that form bioresorbable degradation products by hydrolysis of ester groups are CSA-4108, CSA-4110(racemic mixture), CSA-4110R (R-enantiomer), CSA-4110S (S-enantiomer), CSA-4112, CSA-4114, and salts thereof:

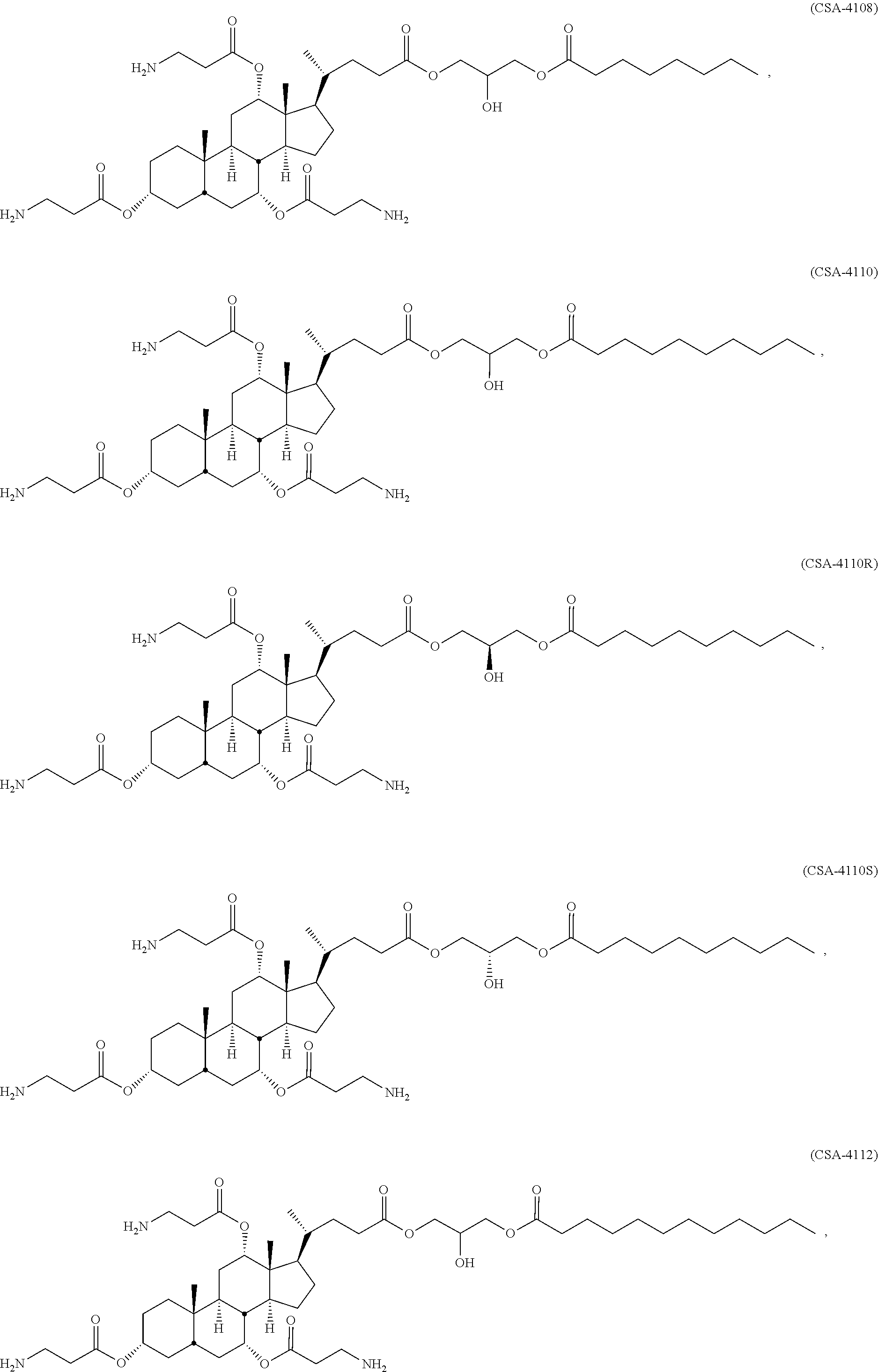

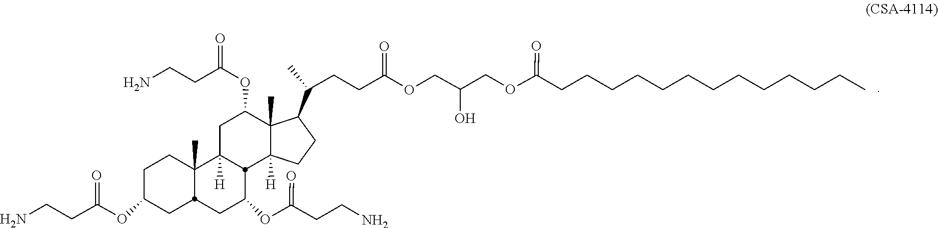

Non-limiting examples of bioresorbable diglyceride CSA compounds that form bioresorbable degradation products by hydrolysis of ester groups are CSA-4204, CSA-4206, CSA-4208, CSA-4210, and CSA-4310, and salts thereof:

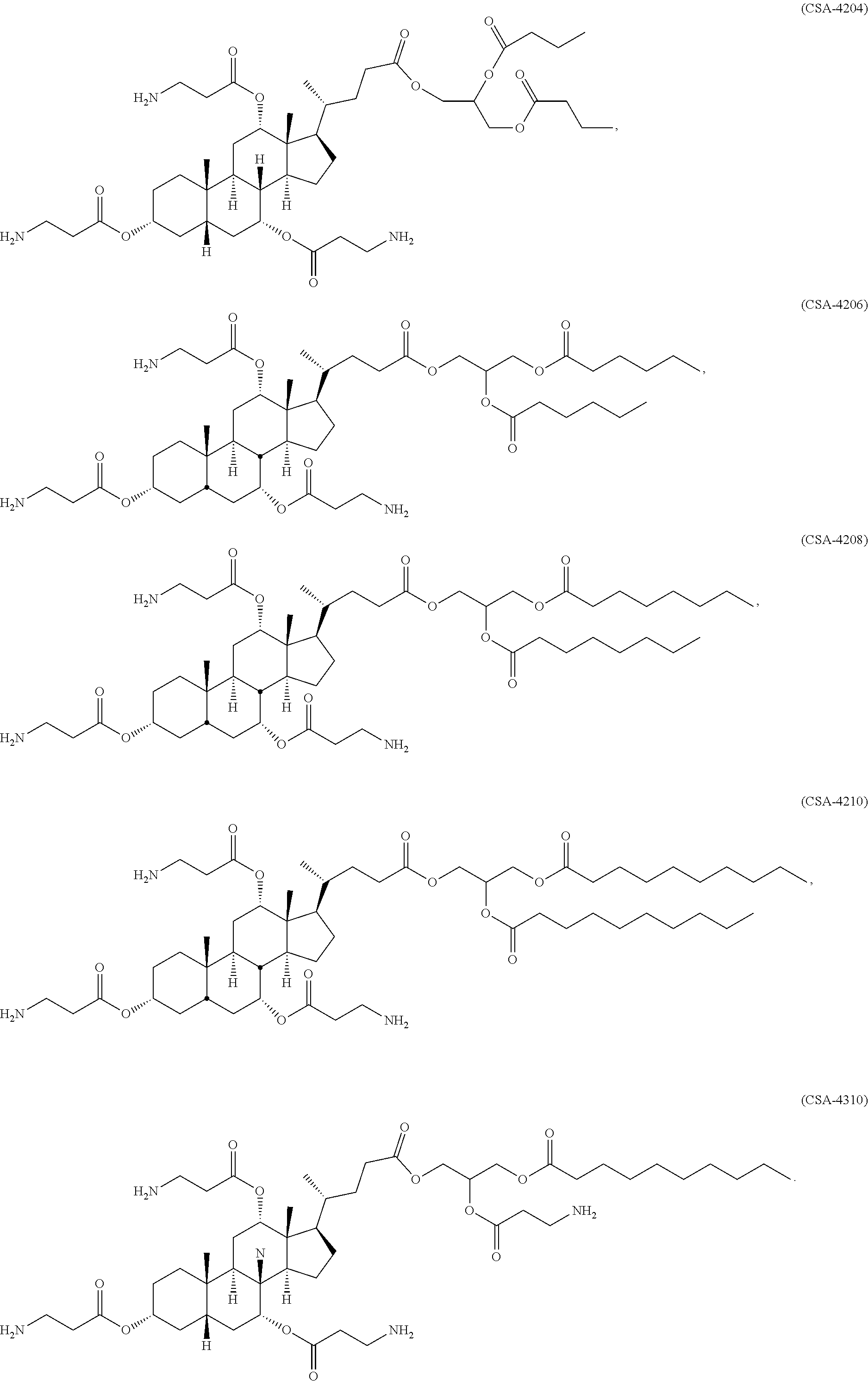

The degradation products of CSA-4108, CSA-4110, CSA-4110R, CSA-4110S, CSA-4112, CSA-4114, CSA-4204, CSA-4206, CSA-4208, CSA-4210, CSA-4310, and salts thereof. comprise a plurality of the following fully bioresorbable molecules:

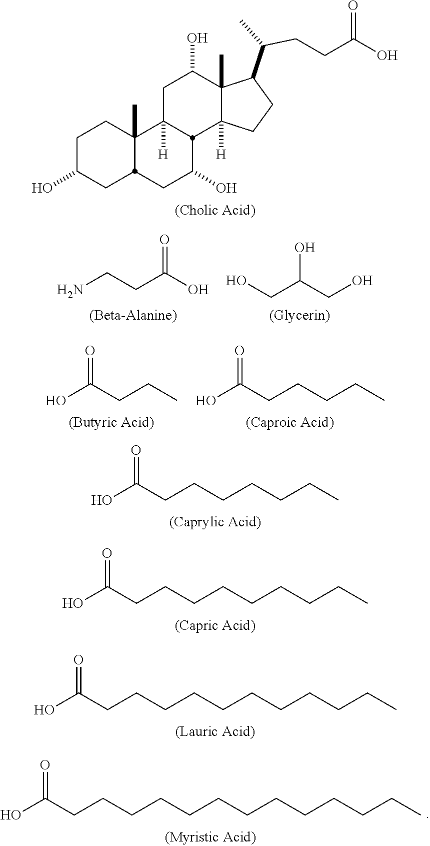

In other embodiments, the fatty acid ester group(s) attached at the C24 position in R₁₈ by a glyceride linkage can be replaced with any other fatty acid, such as one or more of (C₂-C₅) small chain fatty acids, (C₆-C₁₂) medium chain fatty acids, (C12-C26) long chain fatty acids, monounsaturated fatty acids, and polyunsaturated fatty acids.

In other embodiments, the amino acid ester groups of R₃, R₇, and R₁₂ can be replaced with any other amino acid ester groups, such as those based on the example amino acids set forth herein.

### Pharmaceutical Compositions

While CSA compounds described herein can be administered alone, it may be preferable to formulate the compounds as pharmaceutical compositions (i.e., formulations). A pharmaceutical composition is any composition that may be administered in vitro or in vivo or both to a subject in order to treat or ameliorate a condition. In a preferred embodiment, a pharmaceutical composition may be administered in vivo. A subject may include one or more cells or tissues, or organisms. In exemplary embodiments, the subject is an animal. In embodiments, the animal is a mammal. The mammal may be a human or primate in some embodiments. A mammal includes any mammal, such as by way of non-limiting example, cattle, pigs, sheep, goats, horses, camels, buffalo, cats, dogs, rats, mice, and humans.

“Pharmaceutically acceptable” and “physiologically acceptable” mean a biologically compatible formulation, gaseous, liquid or solid, or mixture thereof, which is suitable for one or more routes of administration, in vivo delivery, or contact. A formulation is compatible in that it does not destroy activity of an active ingredient therein (e.g., a CSA compound), or induce adverse side effects that far outweigh any prophylactic or therapeutic effect or benefit.

Pharmaceutical compositions may be formulated with a pharmaceutically acceptable excipient, such as a carrier, solvent, stabilizer, adjuvant, diluent, etc., depending upon the particular mode of administration and dosage form. The pharmaceutical compositions can be formulated to achieve a physiologically compatible pH, and may range from about 3 to 11, preferably about 3 to 7, depending on the formulation and route of administration. In alternative embodiments, the pH is adjusted to about 5 to 8. The pharmaceutical compositions may comprise a therapeutically or prophylactically effective amount of at least one compound as described herein, together with one or more pharmaceutically acceptable excipients.

The pharmaceutical composition may comprise a combination of compounds described herein and/or may include a second active ingredient useful in the treatment or prevention of bacterial infection (e.g., anti-bacterial or anti-microbial agents).

The composition can be formulated as a coating, such as on a medical device. In embodiments, the coating is on a medical instrument.

Formulations for parenteral or oral administration can be solids, liquid solutions, emulsions or suspensions. Inhalable formulations for pulmonary administration can be liquids or powders. A pharmaceutical composition can be formulated as a lyophilized solid that is reconstituted with a physiologically compatible solvent prior to administration. Alternative pharmaceutical compositions may be formulated as syrups, creams, ointments, tablets, etc.

Compositions may contain one or more excipients. Pharmaceutically acceptable excipients are determined in part by the particular composition being administered as well as by the particular method used to administer the composition. There exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are pharmaceutically acceptable excipients.

Pharmaceutical compositions may be formulated in any form suitable for the intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

Pharmaceutical compositions can be formulated as a suspension comprising a CSA compound in admixture with at least one pharmaceutically acceptable excipient suitable for the manufacture of a suspension.

Pharmaceutical compositions can be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcellulose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); polysaccharides and polysaccharide-like compounds (e.g. dextran sulfate); glycoaminoglycans and glycosaminoglycan-like compounds (e.g., hyaluronic acid); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

Pharmaceutical compositions may be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Pharmaceutical compositions may be in the form of a sterile injectable preparation, such as a sterile injectable aqueous emulsion or oleaginous suspension. The emulsion or suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,2-propandiol.

Sterile injectable preparations may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose, any bland fixed oil may be employed including synthetic mono- or diglycerides. In addition, fatty acids such as oleic acid may likewise be used in the preparation of injectables.

To obtain a stable water-soluble dose form of a pharmaceutical composition, a pharmaceutically acceptable salt of a compound described herein may be dissolved in an aqueous solution of an organic or inorganic acid, such as 0.3 M solution of succinic acid, or more preferably, citric acid. If a soluble salt form is not available, the compound may be dissolved in a suitable co-solvent or combination of co-solvents. Examples of suitable co-solvents include alcohol, propylene glycol, polyethylene glycol 300, polysorbate 80, glycerin and the like in concentrations ranging from about 0 to 60% of the total volume. In one embodiment, the active compound is dissolved in DMSO and diluted with water.

Pharmaceutical composition may also be in the form of a solution of a salt form of the active ingredient in an appropriate aqueous vehicle, such as water or isotonic saline or dextrose solution. Also contemplated are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, and complexation.

Many therapeutics have undesirably short half-lives and/or undesirable toxicity. Thus, the concept of improving half-life or toxicity is applicable to various treatments and fields. Pharmaceutical compositions can be prepared, however, by complexing the therapeutic with a biochemical moiety to improve such undesirable properties. Proteins are a particular biochemical moiety that may be complexed with a CSA for administration in a wide variety of applications. In some embodiments, one or more CSAs are complexed with a protein. In some embodiments, one or more CSAs are complexed with a protein to increase the CSA's half-life. In other embodiments, one or more CSAs are complexed with a protein to decrease the CSA's toxicity. Albumin is a particularly preferred protein for complexation with a CSA. In some embodiments, the albumin is fat-free albumin.

With respect to the CSA therapeutic, the biochemical moiety for complexation can be added to the pharmaceutical composition as 0.25, 0.5, 0.75, 1, 1.5, 2, 2.5, 3, 3.5, 4, 4.5, 5, 10, 20, 50, or 100 weight equivalents, or a range bounded by any two of the aforementioned numbers, or about any of the numbers. In embodiments, the weight ratio of albumin to CSA is about 18:1 or less, such as about 9:1 or less. In embodiments, the CSA is coated with albumin.

Non-biochemical compounds can be added to the pharmaceutical compositions to reduce the toxicity of the therapeutic and/or improve the half-life. Suitable amounts and ratios of an additive that can reduce toxicity can be determined via a cellular assay. With respect to the CSA therapeutic, toxicity reducing compounds can be added to the pharmaceutical composition as 0.25, 0.5, 0.75, 1, 1.5, 2, 2.5, 3, 3.5, 4, 4.5, 5, 10, 20, 50, or 100 weight equivalents, or a range bounded by any two of the aforementioned numbers, or about any of the numbers. In embodiments, the toxicity reducing compound is a cocoamphodiacetate such as Miranol® (disodium cocoamphodiacetate). In embodiments, the toxicity reducing compound is an amphoteric surfactant. In embodiments, the toxicity reducing compound is a surfactant. In embodiments, the molar ratio of cocoamphodiacetate to CSA is between about 8:1 and 1:1, preferably about 4:1. In embodiments, the toxicity reducing compound is allantoin.

In embodiments, a CSA composition is prepared utilizing one or more sufactants. In specific embodiments, the CSA is complexed with one or more poloxamer surfactants. Poloxamer surfactants are nonionic triblock copolymers composed of a central hydrophobic chain of polyoxypropylene (poly (propylene oxide)) flanked by two hydrophilic chains of polyoxyethylene (poly (ethylene oxide)). In some embodiments, the poloxamer is a liquid, paste, or flake (solid). Examples of suitable poloxamers include those by the trade names Synperonics, Pluronics, or Kolliphor. In some embodiments, one or more of the poloxamer surfactant in the composition is a flake poloxamer. In embodiments, the one or more poloxamer surfactant in the composition has a molecular weight of about 3600 g/mol for the central hydrophobic chain of polyoxypropylene and has about 70% polyoxyethylene content. In embodiments, the ratio of the one or more poloxamer to CSA is between about 50 to 1; about 40 to 1; about 30 to 1; about 20 to 1; about 10 to 1; about 5 to 1; about 1 to 1; about 1 to 10; about 1 to 20; about 1 to 30; about 1 to 40; or about 1 to 50. In embodiments, the ratio of the one or more poloxamer to CSA is between 50 to 1; 40 to 1; 30 to 1; 20 to 1; 10 to 1; 5 to 1; 1 to 1; 1 to 10; 1 to 20; 1 to 30; 1 to 40; or 1 to 50. In embodiments, the ratio of the one or more poloxamer to CSA is between about 50 to 1 to about 1 to 50. In embodiments, the ratio of the one or more poloxamer to CSA is between about 30 to 1 to about 3 to 1. In some embodiments, the poloxamer is Pluronic F127.

The amount of poloxamer may be based upon a weight percentage of the composition. In embodiments, the amount of poloxamer is about 10%, 15%, 20%, 25%, 30%, 35%, 40%, about any of the aforementioned numbers, or a range bounded by any two of the aforementioned numbers or the formulation. In embodiments, the one or more poloxamer is between about 10% to about 40% by weight of a formulation administered to the patient. In some embodiments, the one or more poloxamer is between about 20% to about 30% by weight of the formulation. In embodiments, the formulation contains less than about 50%, 40%, 30%, 20%, 10%, 5%, or 1% of CSA. In embodiments, the formulation containes less than about 20% by weight of CSA. The above described poloxamer formulations are particularly suited for the methods of treatment, device coatings, preparation of unit dosage forms (i.e., solutions, mouthwashes, injectables), etc.

In embodiments, the compounds described herein may be formulated for oral administration in a lipid-based formulation suitable for low solubility compounds. Lipid-based formulations can generally enhance the oral bioavailability of such compounds.

A pharmaceutical composition may comprise a therapeutically or prophylactically effective amount of a compound described herein, together with at least one pharmaceutically acceptable excipient selected from the group consisting of medium chain fatty acids or propylene glycol esters thereof (e.g., propylene glycol esters of edible fatty acids such as caprylic and capric fatty acids) and pharmaceutically acceptable surfactants such as polyoxyl 40 hydrogenated castor oil.

In embodiments, cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. A particularly preferred cyclodextrin solubility enhancer is hydroxypropyl-o-cyclodextrin (BPBC), which may be added to any of the above-described compositions to further improve the aqueous solubility characteristics of the compounds of the embodiments. In one embodiment, the composition comprises about 0.1% to about 20% hydroxypropyl-o-cyclodextrin, more preferably about 1% to about 15% hydroxypropyl-o-cyclodextrin, and even more preferably from about 2.5% to about 10% hydroxypropyl-o-cyclodextrin. The amount of solubility enhancer employed will depend on the amount of the compound of the embodiments in the composition.

### Synthesis

The methods disclosed herein may be as described below, or by modification of these methods. Ways of modifying the methodology include, among others, temperature, solvent, reagents etc., known to those skilled in the art. In general, during any of the processes for preparation disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

An exemplary but non-limiting general synthetic route for preparing compounds of Formula I, Formula II, and Formula III is shown in Scheme 1. Unless otherwise indicated, the variable definitions are as above for Formulae I, II and/or III. The particular CSA compound formed in this example is CSA-4110 but can be adapted to manufacture any CSA compound within the scope of the disclosure and claims.

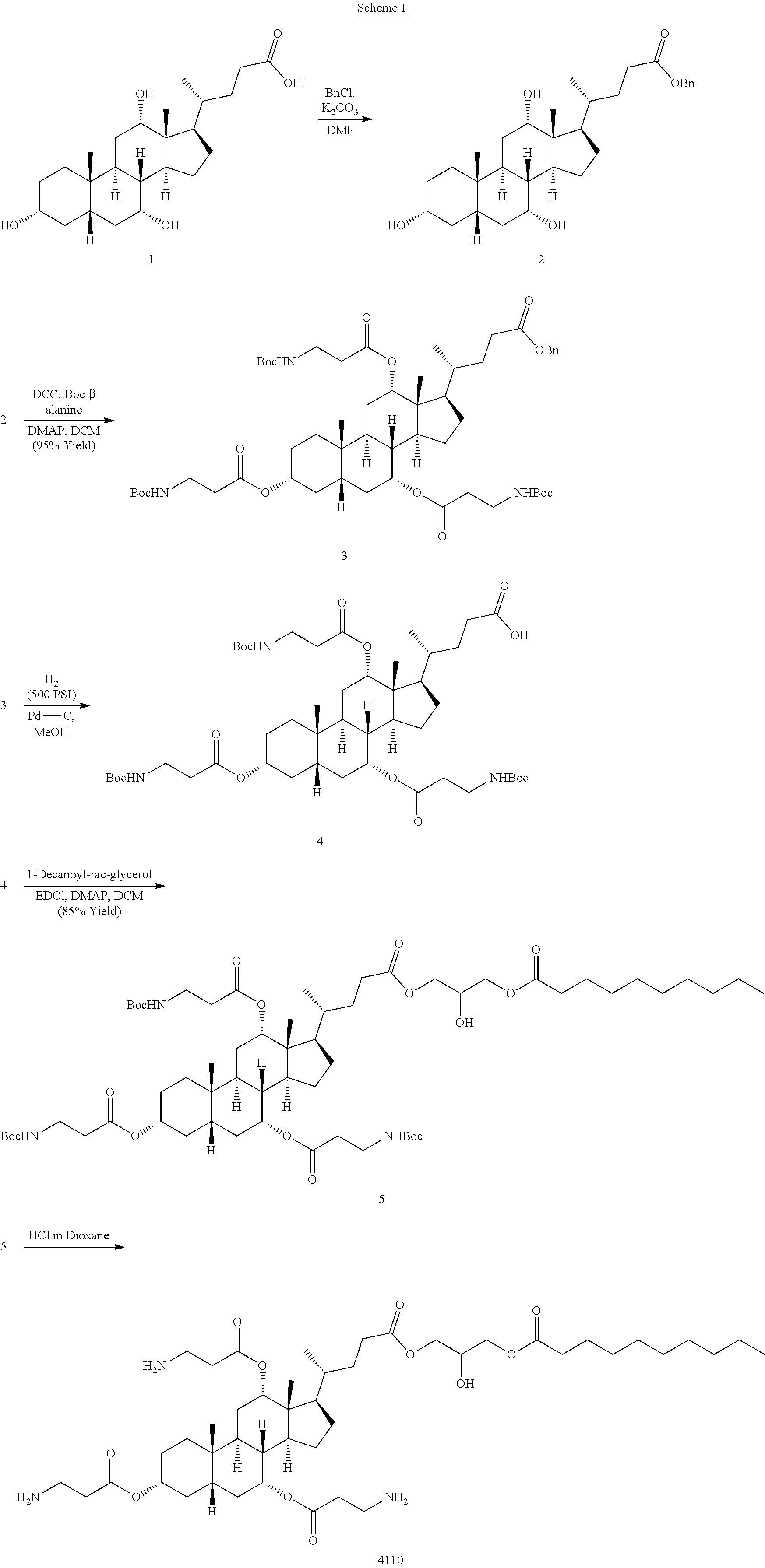

In a first step, cholic acid (1) is reacted with a protecting agent (e.g., benzyl chloride) in a polar aprotic solvent (e.g., dimethylformamide (DMF)) in the presence of a base (e.g., potassium carbonate) to yield protected cholic acid (2) having a protected carboxyl group at the C24 position.

In a second step, the protected cholic acid (2) is reacted with an N-protected amino acid (e.g., beta-alanine protected with tert-butyloxycarbonyl (Boc), fluorenylmethoxycarbony (Fmoc), or benzylchloroformate (Cbz)) via carbodiimide coupling using N,N′-dicyclohexyl-carbodiimide (DCC) in a solvent system containing 4-dimethylaminopyridine (DMAP) and dichloromethane (DCM) to yield intermediate compound (3), or analog thereof, having N-protected aminoalkylcarboxy ester groups at the C3, C7 and C12 positions of the sterol backbone and the protected carboxyl group at the C24 position.

The intermediate compound (3), or analog thereof, is reacted with hydrogen gas (e.g., at a pressure of 500 psi) in the presence of a palladium catalyst on carbon (Pd-C) in methanol (MeOH) to deprotect the protected carboxyl group at the C24 position and yield intermediate compound (4), or analogue thereof, having an unprotected carboxyl group at the C24 position.

The intermediate compound (4), or analogue thereof, is reacted with 1-decanoyl-rac-glycerol via carbodiimide coupling using 1-ethyl-3-(3-dimethylaminopropyl) carbodiimide (EDCI) in a solvent system containing 4-dimethylaminopyridine (DMAP) and dichloromethane (DCM) to form a glyceride ester at the C24 position and yield intermediate compound (5), or analog thereof.

The N-protected aminoalkylcarboxy ester groups at the C3, C7 and C12 positions of intermediate compound (5), or analog thereof, are deprotected and acidified with hydrochloric acid (HCl) in dioxane to form the deprotected HCl acid addition salt of CSA-4110, or analog thereof (e.g., CSA-4108, CSA-4112, CSA-4114, CSA-4206, CSA-4208, or acid addition salt thereof).

The HCl acid addition salt of CSA-4110, or analog thereof, can be purified and optionally neutralized with a base, followed by separation (e.g., 2-phase liquid extraction followed by evaporation of organic solvent) to yield the purified free base. The free base can be used as is or it can be acidified with any desired acid to form an acid addition salt.

An example acid addition salt is the salt of 1,5-naphthalenedisulfonic acid (1,5-NDSA salt, e.g., di-addition salt), which has lower solubility in water than the HCl acid addition salt. The lower water solubility of the NDSA salt of the CSA compound can facilitate further washing and purification to yield an even more pure CSA compound. In addition, the lower water solubility can make the CSA compound useful as a coating, such as a coating on an implantable medical device.

Making the CSA compound ionic, such as by exchanging the NDSA portion with other anions, e.g., chloride ions using HCl, sodium chloride, etc., results in first order release kinetics, which can be accelerated in acidic conditions.

An exemplary but non-limiting synthetic route for preparing 1-decanoyl-rac-glycerol, a mono-glyceride of capric (decanoic) acid, or other mono-or di-glyceride fatty acid analogs, for use in forming the glyceride constituent of R₁₈ is shown in Scheme 2. The particular glyceride formed in this example is mono-glyceride of capric (decanoic) acid but can be adapted to manufacture any mono-or diglyceride of any fatty acid(s) within the scope of the disclosure and claims.

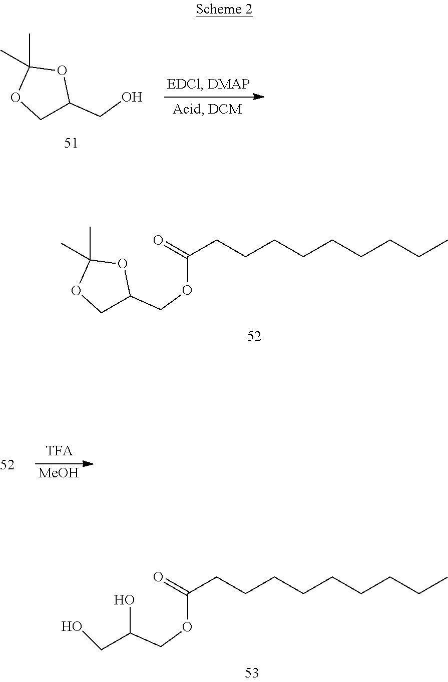

Isopropylidene glycerol (51), also known as solketal or 2,2-dimethyl-1,3-dioxolan-4-yl) methanol, is reacted with a fatty acid (e.g., decanoic (capric) acid) via carbodiimide coupling using 1-ethyl-3-(3-dimethylaminopropyl) carbodiimide (EDCI) in a solvent system containing 4-dimethylaminopyridine (DMAP) and dichloromethane (DCM) to form intermediate compound (52), or analog thereof.

The intermediate compound (52), or analog thereof, is reacted with trifluoroacetic acetic acid (TFA) in methanol (MeOH) to yield compound (53), which is the mono-glyceride of decanoic (capric) acid, or analog thereof.

In some embodiments, the foregoing steps can be performed in a one-pot reaction without purification of intermediate compounds. In some embodiments, intermediate compound (52) is purified before using it to make the glyceride intermediate (53), or analog thereof, used to make the inventive CSA mono-or diglycerides disclosed herein.

An advantage of the disclosed bioresorbable CSA compounds is that they degrade into endogenous compounds, such as cholic acid, amino acid, glycerin, and fatty acid.

An essentially similar synthetic route for preparing CSA compounds with specific mono- and diglyceride linkages, is shown in Schemes 3a and 3b. Scheme 3a yields various monoglycerides of varying chain length at the C24 position, and Scheme 3b yields diglycerides of varying chain length at the C24 position,

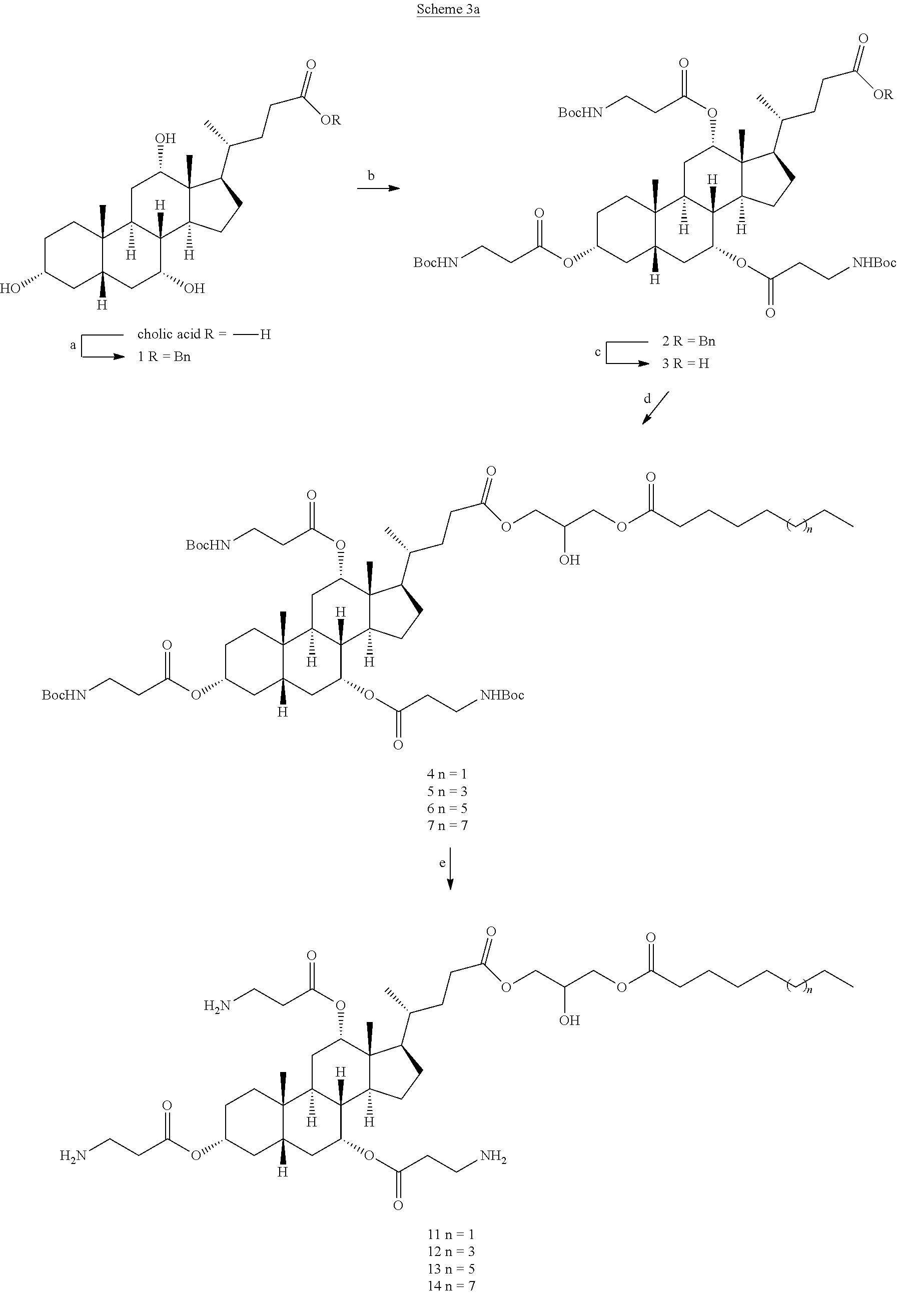

Compounds 11-14 correspond to CSA-4108, CSA-4110, CSA-4112, and CSA-4114, respectively. It shall be understood, however, that Scheme 3a can be modified to produce monoglycerides of any chain length (c.g., using fatty acids with 2-22 carbons).

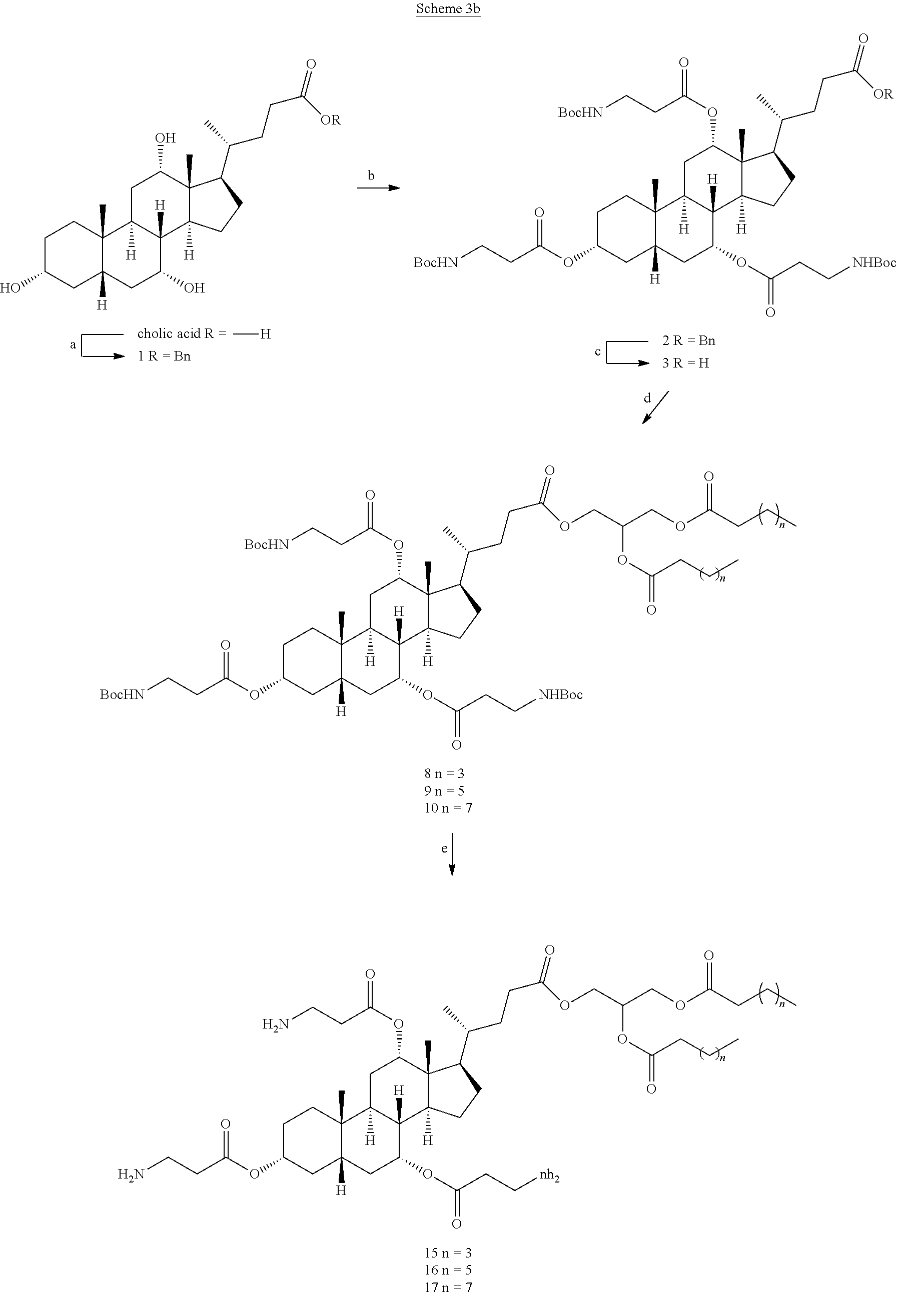

Compounds 15-17 correspond to CSA-4206, CSA-4208, and CSA-4210, respectively. It shall be understood, however, that Scheme 3b can be modified to produce diglycerides of any chain length (e.g., using fatty acids with 2-22 carbons). Scheme 3b can also be modified to yield mixed diglycerides, such as CSA-4310, by using a mixed diglyceride intermediate that includes an alkyl ester and an N-protected aminoalkyl ester, which can be deprotected at the same time that the N-protected aminoalkyl esters groups at the C3, C7 and C12 positions are deprotected.

In both Schemes 3a and 3b, the reactions in steps (a) through (e) depicted above use the following materials: Step (a): BnCl, K₂CO₃, DMF (86%); Step (b) DCC, Boc β-alanine, 4-dimethylaminopyridine (DMAP), dichloromethane (DCM) (95%); Step (c) H₂ (500 PSI), Pd-C, MeOH (92%); Step (d) 1-octanoyl-rac-glycerol, 1-decanoyl-rac-glycerol, 1-lauroyl-rac-glycerol, 1,2-dibutanoyl-sn-glycerol, 1,2-dihexanoyl-sn-glycerol, 1,2-dioctanoyl-sn-glycerol or monomyristin, 1-ethyl-3-carbodiimide (EDCI), DMAP, DCM; Step (e) HCl in dioxane.

Starting from cholic acid, the C24 of cholic acid was esterified using benzyl chloride to yield (1). The hydroxyl groups at C3, C7, and C12 of cholic acid were then esterified with Boc-β-alanine to yield (2). The benzyl ester at C24 was then removed using palladium catalyzed hydrogenation to yield (3). The acid at C24 of (3) was then coupled to various monoglyceride and diglyceride tails.

Synthesis of (3) was scaled up to 20 grams to enable preparation of the seven targeted compounds, including gram quantities of (12) (CSA-4110). The simplicity of the synthetic route is amenable to large-scale production.

Most of the monoglyceride tails were commercially available. The enantiomerically pure forms of the monoglycerides were not. The enantiopure R and S monoglycerides for producing CSA-4110R and CSA-4110S were formed by esterifying solketal using Steglich esterification, deprotecting with TFA, and esterifying to (3) (see Schemes 4a and 4b below). Previous syntheses of enantiopure monocaprin have been reported, but used reagents that were unavailable for purchase at the time of this synthesis or used a synthetic pathway that had more steps.

One of the diglyceride tails was not commercially available. As such, CSA-4204 (21) was produced by esterifying (3) with solketal directly, deprotecting with TFA, esterifying with butanoic acid, and deprotecting with HCl in dioxane (see Scheme 5 below).

Scheme 4a shows a synthetic route for preparing CSA-4110R (25), and Scheme 4b shows a synthetic route for preparing CSA-4110S (29).

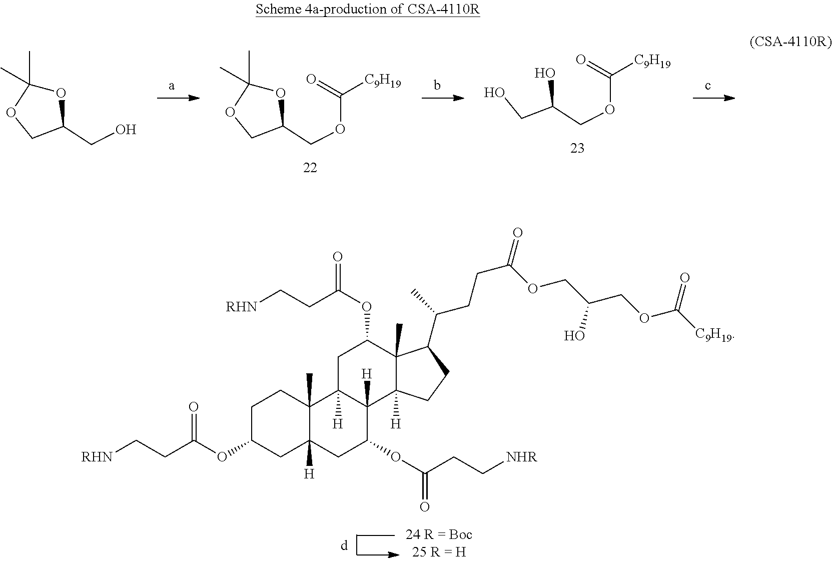

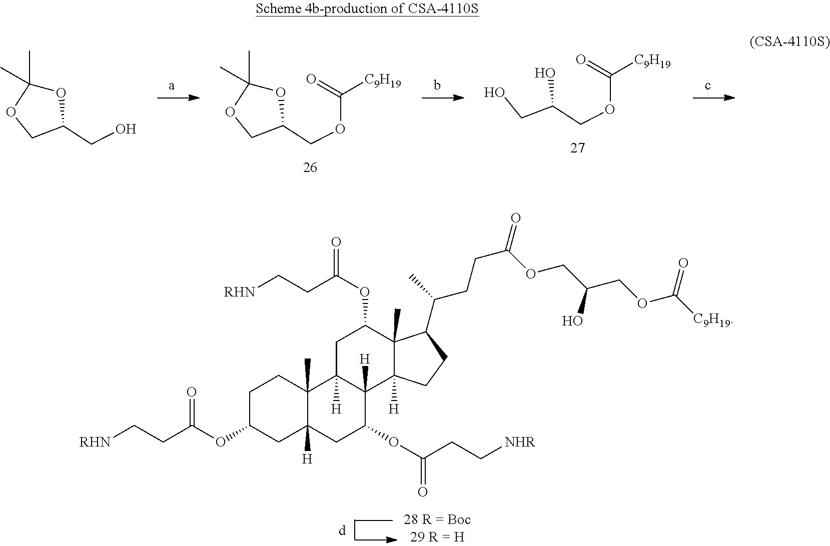

In both Schemes 4a and 4b, the reactions in steps (a) through (d) depicted above use the following materials: Step (a) solketal (chiral alternatives), decanoic acid, EDCI, DMAP, DCM (69%); Step (b) TFA, McOH (48%); Step (c) (23) or (27), (3) from Scheme 3a, EDCI, DMAP, DCM; Step (d) HCl in dioxane.

Scheme 5 shows a synthetic route for preparing CSA 4204 (21).

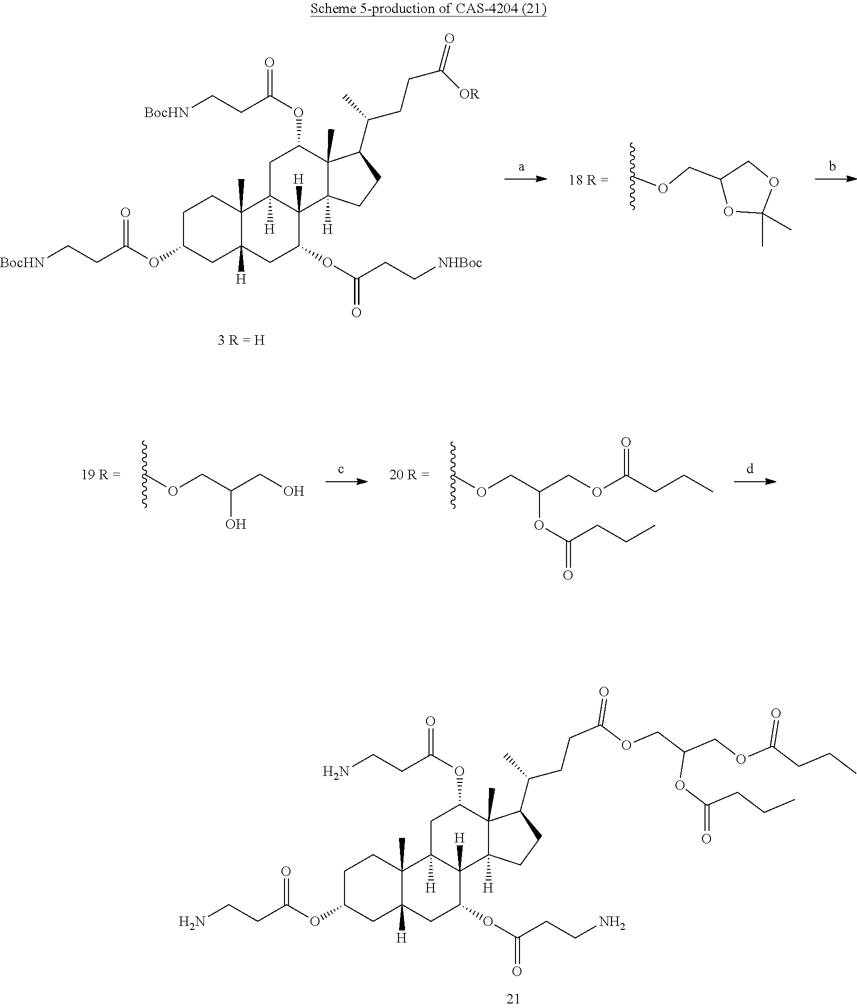

In Scheme 5, the reactions in steps (a) through (d) depicted above use the following materials: Step (a) solketal, EDCI, DMAP, DCM (56%); Step (b) TFA, MeOH (60%); Step (c) butanoic acid, EDCI, DMAP, DCM (70%); Step (d) HCl in dioxane (92%). In this embodiment, (3) was esterified with solketal directly to form (18), deprotected to form (19), which was esterified with butanoic acid to form (20), which was deprotected to form (21) (CSA-4204).

### EXAMPLES

### Example 1

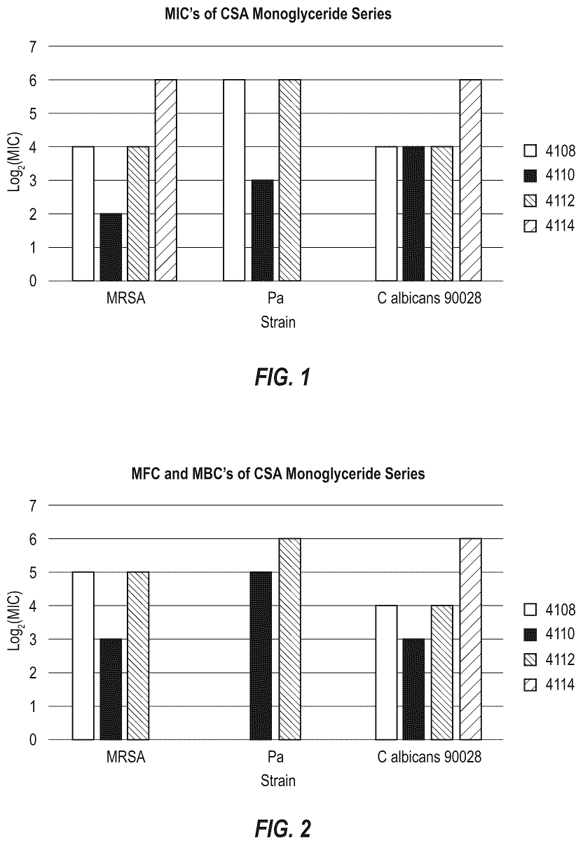

The antimicrobial activities of CSA-4108, CSA-4110, CSA-4112, and CSA-4114 were determined relative to methicillin-resistant Staphylococcus aureus (MRSA), Pseudomonas aeruginosa (Pa), and Candida albicans 90028. The minimum inhibitory concentration (MIC) for CSA-4108, CSA-4110, CSA-4112, and CSA-4114 relative to the foregoing microbes is graphically reported in FIG. 1. The minimum bactericidal concentration (MBC) and minimum fungicidal concentration (MBC) for CSA-4108, CSA-4110, CSA-4112, and CSA-4114 relative to the foregoing microbes are graphically reported in FIG. 2.

As shown in FIG. 1, CSA-4110 had the lowest MIC, and therefore the highest antimicrobial activity, when used against MRSA and Pa, followed by CSA-4108 and CSA-4112. When used against C. albicans, CSA-4108, CSA-4110, and CSA-4112 had the same MIC. CSA-4114 had the highest MIC, and therefore the lowest antimicrobial activity of the four CSA compounds, when used against MRSA and C. albicans. Nevertheless, CSA-4114 still demonstrated excellent antimicrobial activity against MRSA and C. albicans. Although CSA-4114 was not tested against PA, one would reasonably expect CSA-4114 to have excellent antimicrobial activity against PA, but with a higher MIC than CSA-4108, CSA-4110, and CSA-4112.

As shown in FIG. 2, CSA-4110 again had the lowest MBC and MFC, and therefore the highest antimicrobial activity, when used against MRSA, PA, and C. albicans, followed by CSA-4108 and CSA-4112 against MRSA and C. albicans, and by CSA-4112 against PA. CSA-4114 had the highest MFC, and therefore the lowest antimicrobial activity of the four CSA compounds, when used against C. albicans. Nevertheless, CSA-4114 still demonstrated excellent antimicrobial activity against C. albicans. Although CSA-4108 was not tested against PA, one would reasonably expect CSA-4108 to have excellent antimicrobial activity against PA, but with a higher MBC than CSA-4110 and a similar MBC as CSA-4112. Although CSA-4114 was not tested against MRSA and PA, one would reasonably expect it to have excellent antimicrobial activity against MRSA and PA, but with a higher MBC than CSA-4108, CSA-4110, and CSA-4112.

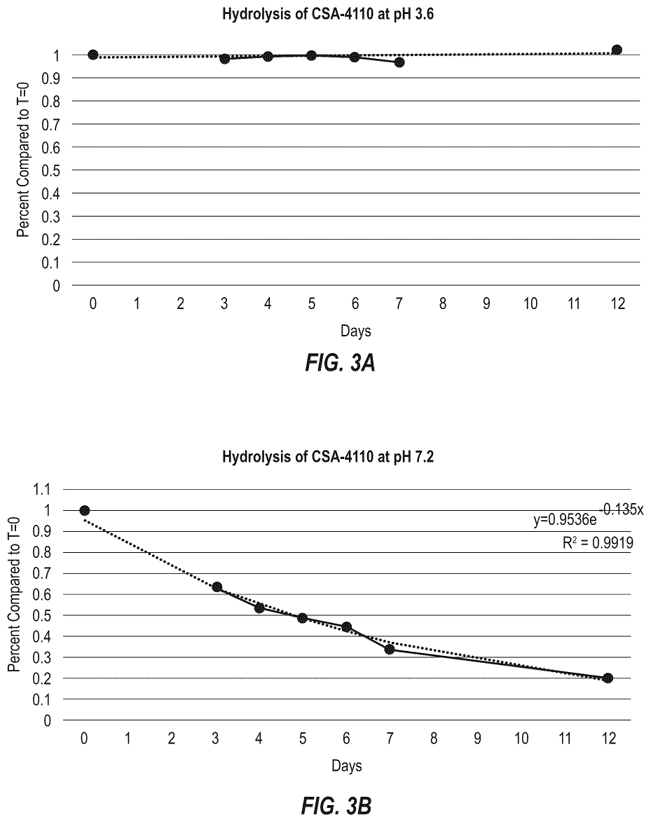

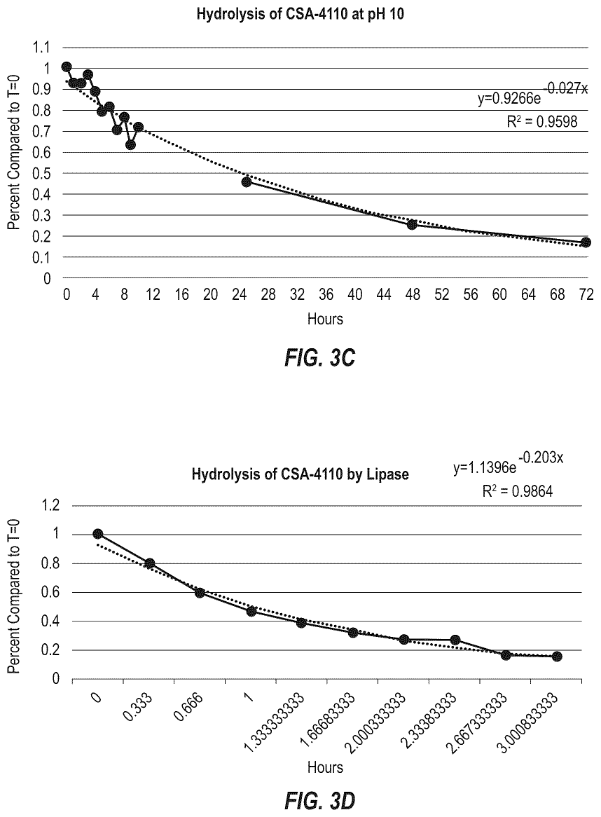

The stability, more particularly the rate of hydrolysis in water, of CSA-4110 was tested at various pH values or when exposed to lipase. The hydrolysis rates of CSA-4110 at the various conditions are graphically reported in FIGS. 3A-3D.

FIG. 3A shows that CSA-4110 resisted hydrolysis, and was therefore highly stable, in an aqueous solution at a pH of 3.6.

FIG. 3B shows that CSA-4110 hydrolyzed more readily, and was therefore less stable, in an aqueous solution at a pH of 7.2.

FIG. 3C shows that CSA-4110 hydrolyzed even more rapidly (in a matter of hours rather than days), and was therefore unstable, in an aqueous solution at a pH of 10.

Finally, FIG. 3D shows that CSA-4110 hydrolyzed most rapidly in an aqueous solution containing lipase.

The data set forth in FIGS. 3A-3D show that CSA-4110, and by extension other mono- and diglycerides of CSA compounds and fatty acids, are quite stable at acidic pH, less stable at neutral pH, and unstable at basic pH, and very quickly hydrolyzed in the presence of lipase, which is found in the body.

### Example 2

Follow up testing of various bioresorbable CSA compounds was performed. As an extension to the previous testing shown in Example 1, bioresorbable CSA compounds were further investigated to determine if and how well they could prevent microbial colonization of bioresorbable implants. The goal was to determine whether a coated, bioresorbable implant would eliminate any microbes that attach themselves to the device or surgical site during implantation. The bioresorbable CSA compound would slowly be released over the course of the following 1-4 weeks, eliminating any planktonic microbes that may attempt to adhere to the device and create a biofilm. The bioresorbable CSA compound released would then be hydrolyzed rapidly into endogenous components, avoiding the steps of oxidation and elimination required with most antimicrobial agents. The expectation is that the bioresorbable device and bioresorbable CSA compound would have comparable duration.

To prepare a CSA compound that is also bioresorbable, the structure of another CSA compound (e.g., CSA-131) was adapted to include endogenous components linked through hydrolysable bonds. The redesign necessarily kept the three key subdomains of the CSA compound structure: 1) a rigid hydrophobic core based on cholic acid, 2) three side chains at C3, C7, and C12 with positive charged groups on each chain that are close together spatially, and 3) a hydrophobic “tail” at C24 with about 8-12 carbons in total. In the case of CSA-131, the hydrophobic core is linked to the side chains by ethers and the tail is linked by an amine. These bond types are stable in water, even at elevated temperatures, and are not readily hydrolyzed by enzymes.

Most bioresorbable materials rely in ester hydrolysis for degradation. For example, polylactic acid-glycolyic acid (PLGA) undergoes spontaneous hydrolysis in water to yield lactic acid and glycolic acid. The ratio of lactic acid to glycolycic acid controls the rate of hydrolysis. The presence of electron- withdrawing oxygen alpha to the ester increases the electrophilicity of the ester and the rate of PLGA hydrolysis.

By incorporating beta-alanine into a CSA compound (see Scheme 6 below), it was anticipated that the beta-ammonium group would increase the electrophilicity of the corresponding esters and promote spontaneous hydrolysis. The antimicrobial activity of CSA compounds is dependent, in large part, by a hydrophobic tail. In the glyceride structure envisioned for bioresorbable CSA compounds, this hydrophobic tail (or tails) would be provided by a fatty acid ester (or esters) on glycerol (see Scheme 6 below). This key design element would allow rapid degradation of the CSA compounds through the action of ubiquitous lipases.

Through lipase activity and spontaneous ester hydrolysis in water, a CSA compound (e.g., CSA-4110 shown in Scheme 6 below) would be degraded to the following endogenous compounds: cholic acid, beta-alanine, glycerin, and a fatty acid (e.g., decanoic acid). Notably, hydrolysis of any one of the esters in this type of ceragenin substantially deactivates the antimicrobial CSA compound.

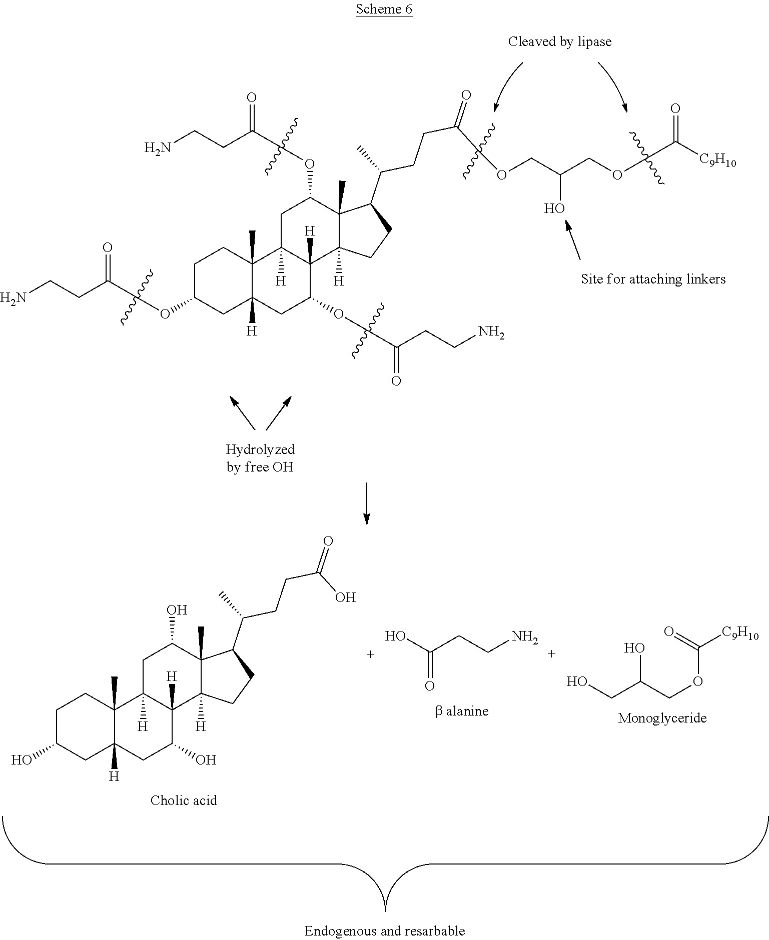

With this ester-based redesign, new variables exist that require structure-function optimization to ensure the best activity against fungi/bacteria. Most of these variables are within the tail region: use of mono-or diglycerides, length of each glyceride, and stereochemistry of the glycerin spacer. Previous work in optimizing the structures of the side chains showed that beta-alanine-containing CSA compounds were more active than those containing glycine or γ-aminobutyric acid. As part of the bioresorbable design constraints for the hydrophobic tail region, efforts were limited to fatty acids containing even numbers of carbons. Targeted CSA compounds were compounds (11) though (17), corresponding to CSA-4108, CSA-4110, CSA-4112, CSA-4114, CSA-4206, CSA-4206, CSA-4208, and CSA-4210, respectively (see Schemes 3a and 3b above).

To investigate the antimicrobial impact of structural variance in the novel bioresorbable CSA compounds disclosed herein,Pseudomonas aeruginosa 01 (PA01), methicillin-resistant Staphylococcus aureus (MRSA), and Candida albicans (CA) were selected, representing Gram-negative bacterial, Gram-positive bacterial, and fungal pathogens of high or critical priority on the world health organization's priority lists for bacteria and fungi. Earlier applications of CSA compounds have focused on preventing medical device infections, and all three strains are commonly found in colonized implants, making efficacy against these pathogens an important indicator of future applicability.

All compounds were screened against these pathogens to determine their minimum inhibitory concentration (MIC), minimum bactericidal concentration (MBC), and minimum fungicidal concentration (MFC) using a broth microdilution method. MRSA was found to be the most susceptible, and among bioresorbable CSA compounds with different tail lengths, CSA-4110, CSA-4206, and CSA-4204 were inhibitory for visible growth at concentrations ≤8 μg/mL (see Table 1 below). Of these three, only CSA-4110 was similarly active against PA01and CA, inhibiting growth at 8 μg/mL. CSA-4108, CSA-4112, CSA-4114, CSA-4208, and CSA-4210 did not meet this threshold, and comparison of the MICs of CSA-4112 relative to CSA-4114, and CSA-4208 relative to CSA-4210, demonstrates a negative correlation between tail length and activity. The loss of activity with longer compounds with longer tail lengths suggests that a loss in solubility may impact activity. Activities of CSA-4108 and CSA-4112 bracket that of CSA-4110, suggesting that the optimal tail length is derived from decanoic acid. With the bioresorbable CSA compounds containing diglycerides, incorporation of carboxylic acids longer than butyric acid resulted in losses of activity.

MBC data aligned closely with the trends observed in the MIC assays, with CSA-4110, CSA-4206, and CSA-4204 killing at least 99.9% of MRSA at 8 μg/mL, while CSA-4108, CSA-4112, CSA-4114, CSA-4208, and CSA-4210 required at least fourfold concentrations to reach the target threshold. MFC values against CA were 16, 8 and 32 μg/mL, and MBC values against PA01 were 32, 32 and 100, for CSA-4110, CSA-4206, and CSA-4204, respectively (see Table 1 below). Effective concentrations for the other five compounds were generally higher than CSA-4110 and CSA-4206, though CSA-4108, CSA-4110, and CSA-4112 were all effective against CA at 16 μg/mL. Notably, the ratio of MBC/MFC to MIC is ≤ 4, indicating these compounds are indeed bactericidal and fungicidal, and are unlikely to keep pathogens in a dormant state in future applications.

TABLE 1

| | MIC (MBC or MFC) | | |
|---|---|---|---|
| Compound | MRSA | PA01 | CA |
| CSA-4108 | 16 (32) | 64 (>100) | 16 (16) |
| CSA-4110 | 4 (8) | 8 (32) | 8 (16) |
| CSA-4112 | 16 (32) | 64 (64) | 16 (16) |
| CSA-4114 | 64 (>100) | >100 (>100) | 64 (64) |
| CSA-4206 | 8 (8) | 32 (32) | 8 (8) |
| CSA-4208 | 32 (64) | 64 (>100) | 64 (64) |
| CSA-4210 | >100 (>100) | >100 (>100) | >100 (>100) |
| CSA-4204 | 4 (8) | 64 (>100) | 16 (32) |
| CSA-4110R | 4 (4) | 4 (8) | 8 (8) |
| CSA-4110S | 4 (4) | 8 (16) | 8 (8) |

Based on the MIC values obtained and because CSA-4110 would possibly allow for linker attachments, CSA-4110 was selected for further structure optimization, and new compounds were synthesized with R (CSA-4110R) and S (CSA-411S) configurations at the glycerin alcohol chiral position to determine if the configuration at this position would have an impact on antimicrobial activity. Both compound CSA-4110R and compound CSA-4110S performed comparably to CSA-4110 (racemic mixture) in all MIC and MBC/MFC assays, indicating a non-specific interaction may be key in the mechanism-of-action.

### Activity of Lead Candidate on Critical Pathogens

CSA-4110 was evaluated further against a broader range of bacterial and fungal strains. Acinetobacter baumannii and several isolates of Staphylococcus aureus, Klebsiella pneumoniae, and Candida auris were selected, further broadening the coverage of high and critical priority pathogens. Streptococcus uberis, Staphylococcus pseudintermedius, Proteus mirabilis, and Escherichia coli were also screened to obtain a more complete efficacy profile. With one notable exception, all MICs obtained were within a range of 2-8 μg/mL and all MBCs/MFCs were within a range of 2-16 μg/mL, indicating that CSA-4110 is capable of being used to control a broad range of pathogens (see Tables 2-4 below). For comparison, previous work established the MICs of CSA-131 against strains of Staphylococcus aureus, Pseudomonas aeruginosa, and Candida albicans at 0.5-2 μg/mL

The one exception to the MIC and MBC ranges is Proteus mirabilis (PM), with an MIC of 16 μg/mL and an MBC of 64 μg/mL. PM is a Gram-negative organism which is occasionally found in urinary tract infections in individuals with functional or anatomical anomalies. It is often noted for its ability to modulate its susceptibility to cationic biocides primarily through modifications to its efflux system and the structure of lipopolysaccharide, a negatively charged endotoxin located in the outer membrane. This can provide insight into the mechanism-of-action for CSA-4110. Given the lack of chiral specificity for its activity in our initial microbial assays and the ability of PM to hide charged components of its lipopolysaccharide (LPS), compound CSA-4110 likely interacts generally with the charged phosphate of LPS to associate with the membrane of pathogens.

TABLE 2

| Gram Positive Bacteria | MIC | MBC or MFC |
|---|---|---|
| S. aureus ATCC 25923 | 4 | 8 |
| S. aureus ATCC 6538 | 4 | 8 |
| S. aureus ATCC 27734 | 4 | 4 |
| S. uberis ATCC 27958 | 2 | 4 |
| S. psuedintermedius ATCC 49444 | 4 | 4 |
| MRSA ATCC BAA-42 | 2 | 2 |

TABLE 3

| Gram Negative Bacteria | MIC | MBC or MFC |
|---|---|---|
| P. mirabilis ATCC 29906 | 16 | 64 |
| P. aeruginosa ATCC 27853 | 4 | 8 |
| P. aeruginosa 01 ATCC 47085 | 4 | 8 |
| K. pneumoniae ATCC BAA-1706 | 4 | 4 |
| K. pneumoniae ARLG 1360 | 8 | 8 |
| K. pneumoniae ATCC 13883 | 2 | 2 |
| E. coli ATCC 25922 | 4 | 4 |
| A. baumannii ATCC 19606 | 4 | 4 |

TABLE 4

| Fungi | MIC | MBC or MFC |
|---|---|---|
| C. albicans ATCC 90028 | 4 | 16 |
| C. auris CDC 381 | 8 | 8 |
| C. auris CDC 382 | 8 | 16 |
| C. auris CDC 383 | 8 | 16 |
| C. auris CDC 384 | 4 | 16 |
| C. auris CDC 385 | 8 | 16 |
| C. auris CDC 386 | 8 | 16 |
| C. auris CDC 387 | 8 | 16 |
| C. auris CDC 388 | 8 | 16 |
| C. auris CDC 389 | 8 | 16 |
| C. auris CDC 390 | 8 | 16 |

### Stability of CSA-4110

To measure the stability of CSA-4110 under various conditions (pH and presence of lipase), monitored degradation over time by HPLC. To enable UV/visible detection, CSA-4110 required derivatization with a chromophore. A precolumn derivatization method was adapted using Fmoc-Cl. This method also required CSA-131 as an internal standard to eliminate errors due to volumetric variations.

With a reliable quantitative analysis method in hand, the optimal storage conditions were determined for CSA-4110 in solution and how quickly CSA-4110 degrades when exposed to mammalian lipases and varied pH. Reference is again made to FIGS. 3A-3D. FIG. 3A shows the stability of CSA-4110 at pH 3.6. FIG. 3B shows the stability of CSA-4110 at pH 7.2. FIG. 3C shows the stability of CSA-4110 at pH 10. FIG. 3D shows the stability of CSA-4110 in the presence of lipase.

CSA-4110 is most stable when stored under slightly acidic conditions; at pH 3.6, no measurable degradation was observed after 12 days at room temperature. In contrast, at pH of 7.2 or 10, the half-lives at room temperature were 4.5 days and 24 hours, respectively. When dissolved with a lipase at room temperature at pH=6.9, CSA-4110 had a half-life of 1 hour.

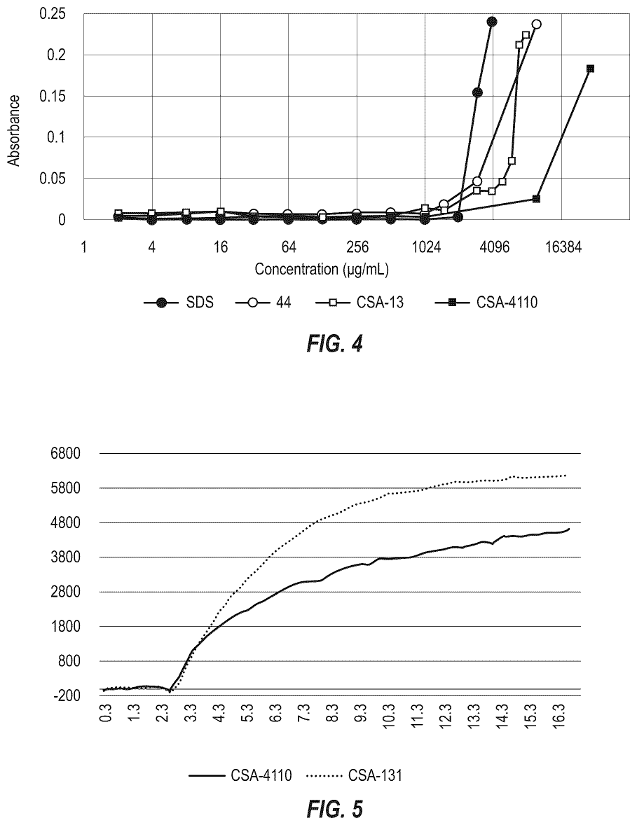

Reference is made to FIGS. 4 and 5. FIG. 4 shows the effects of various concentrations of sodium dodecyl sulfate (SDS), CSA-44, CSA-13, and CSA-4110 on absorbance of orange OT dye. FIG. 5 shows the effects of CSA-131 or CSA-4110 on the fluorescence intensity of the dye DiSC2 (5) in the presence of MRSA ATCC BAA-42. CSA-131 or CSA-4110 was added at 140 second to achieve 8 μg/mL after addition.

### Mechanism of Action

Previous studies have shown membrane disruption and permeabilization to be the mechanism of action of other CSA compounds. The critical micelle concentration (CMC) of CSA-4110 was measured to determine if aggregation was necessary for antimicrobial activity (see FIG. 4). This assay involves using a hydrophobic dye (orange OT) that is insoluble in water to measure what concentration of CSA-4110 is required to solubilize orange OT. Entry of orange OT into solution is due to micelle formation by CSA-4110 and can be quantified with a spectrophotometer. The CMC of CSA-4110 is comparable to sodium dodecyl sulfate (SDS) and other CSA compounds, with solubilization of orange OT occurring near 1 mg/mL. This CMC is orders of magnitude higher than CSA-4110′s MICs of 2-8 g/mL, which suggests that aggregation into micelles is not required for antimicrobial activity.

Antimicrobial peptides (AMPs) and CSA compounds are known to permeabilize bacterial membranes. For bacterial survival, the cytoplasmic membrane must be polarized. Depolarization of this membrane prevents ATP synthesis and leads to cell death. The dye 3,3′-diethylthiadicarbocyanine iodide (DiSC2(5)) can be used to measure cell membrane polarization. Fluorescence of this dye is decreased when it inserts into polarized membranes, and membrane depolarization results in substantial increases in fluorescence (see FIG. 5).

MRSA was used to explore the impact of CSA-4110 on membrane depolarization and compared results to those with ceragenin CSA-131. Incubation of a culture of cells in the presence of the dye resulted in incorporation of the dye in the cytoplasmic membrane. Fluorescence was monitored over time after addition of CSA-4110 or CSA-131. which incorporated into cellular membranes. Addition of either CSA-131 or CSA-4110 resulted in a rapid increase of fluorescence, indicating both are active on bacterial membranes, resulting in loss of polarity. Interestingly, CSA-131 depolarizes the MRSA cells more rapidly than CSA-4110, suggesting a higher efficiency for the former, consistent with the slightly lower effective concentrations observed for CSA-131.

### Conclusions

A series of novel bioresorbable CSA compounds were designed, synthesized, and assessed for their antibacterial and antifungal activity. From this series, ceragenin CSA-4110 is noted for its MICs of 2-8 μg/mL against several high and critical priority pathogens such as A. baumannii, S. aureus, K. pneumoniae, and C. auris. The degradation of CSA-4110 under physiologically relevant pH's, in storage solution, and in presence of lipase was studied, and half-lives of CSA-4110 in each set of conditions were determined. CSA-4110 is a substrate for lipase, and esters in the molecule spontaneous hydrolyze at basic and neutral pH. In contrast, CSA-4110 is relatively stable under acidic pH. The mechanism of action of CSA-4110 is unrelated to micelle formation and is due, at least in part, to cytoplasmic membrane depolarization. Overall, CSA-4110 is favorable for use in medical device coatings given its broad-spectrum antimicrobial activity and that it will rapidly degrade after eluting from a coating. That is, CSA-4110 will exert antimicrobial activity at the device surface without extending this activity into surrounding tissues. Thus, biofilm formation will be prevented on a device surface while minimizing systemic exposure and interaction with the natural microfluora.

Although CSA-4110 was found to perform better than the other bioresorbable CSA compounds tested and described herein, the other bioresorbable CSA compounds show similar or adequate antimicrobial activity and are therefore suitable candidates as effective antimicrobial agents, such as in the same or other contexts.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A bioresorbable cationic steroidal antimicrobial (CSA) compound having a structure of Formula I or II, or a salt thereof: 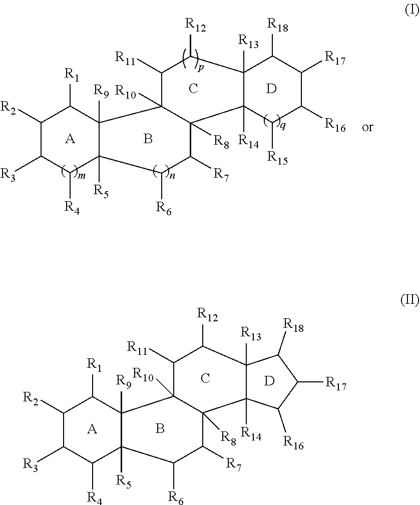
where,
m, n, p, and q are independently 0 or 1;
rings A, B, C, and D are independently saturated, or are fully or partially unsaturated, provided that at least two of rings A, B, C, and D are saturated;
R₁ through R₁₈ are independently selected from the group consisting of hydrogen, hydroxyl, alkyl, hydroxyalkyl, alkyloxyalkyl, alkylcarboxyalkyl, terpenylcarboxyalkyl, terpenylcarbonyloxyalkyl, terpenylamidoalkyl, terpenylaminoalkyl, terpenyloxyoalkyl, alkylaminoalkyl, alkylamino-alkylamino, alkylaminoalkylaminoalkylamino, aminoalkyl, aryl, arylaminoalkyl, haloalkyl, alkenyl, alkynyl, oxo, linking group attached to a second steroid, aminoalkylurethanyl, aminoalkenylurethanyl, aminoalkynylurethanyl, aminoarylurethanyl, aminoalkyloxy, aminoalkylcarboxy, aminoalkyloxyalkyl, aminoalkylaminocarbonyl, aminoalkylcarboxamido, di (alkyl)aminoalkyl, H₂N—HC (Q₅) (C═O)—O—, H₂N—HC (Q₅)-(C═O)—NH—, azidoalkyloxy, cyanoalkyloxy, P.G.-HN—HC (Q₅)-(C═O)—O—, guanidino-alkyloxy, quaternary ammonium alkylcarboxy, and guanidinoalkyl carboxy, where Qs is a side chain of any amino acid (including a side chain of glycine, i.e., H), and P.G. is an amino protecting group; and
R₅, R₈, R₉, R₁₀, R₁₃, R₁₄ and R₁₇ are independently deleted when one of rings A, B, C, or D is unsaturated so as to complete the valency of the carbon atom at that site, provided that at least one of Ri through R₄, R₆, R₇, R₁₁, R₁₂, R₁₅, R₁₆, and R₁₈, preferably
R₁₈, includes a glyceride of a fatty acid attached to the sterol backbone by an ester linkage or amide linkage, and
provided that at least one of Ri through R₄, R₆, R₇, R₁₁, R₁₂, R₁₅, R₁₆, and R₁₈, preferably one, two or three of R₃, R₇, and R₁₂, includes an amino acid attached to the sterol backbone by an ester linkage, amide linkage, or ether linkage.

2. The bioresorbable CSA compound of claim 1, wherein the CSA compound has a structure of Formula III, in which R₁₅ is omitted:

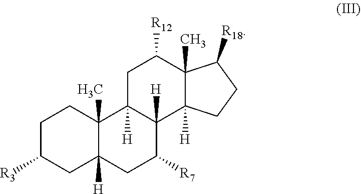

3. The bioresorbable CSA compound of claim 1, wherein at least one of R₁-R₁₈. preferably R₁₈, has the following structure:
—R₁₉—(C═O)—O—C—CR₂₀—CR₂₁
where R₁₉ is omitted or is selected from alkyl, alkenyl, alkynyl, and aryl, and R₂₀ and R₂₁are independently selected from hydroxy and (C₂-C₂₂)alkylcarboxy, provided that at least one of R₂₀ or R₂₁ is (C₂-C₂₂)alkylcarboxy.

4. The bioresorbable CSA compound of claim 1, wherein R₁₈ has the following structure:
—R₁₉—(C═O)—O—C—CR₂₀—CR₂₁
where R₁₉ is omitted or is selected from alkyl, alkenyl, alkynyl, and aryl, Rzo is a (C₂-C₂₂)alkylcarboxy and R₂₁ has the following aminoalkylcarboxy structure:
R₂₄R₂₃N—R₂₂—(C═O)—O—
where R₂₂ is a substituted or unsubstituted alkyl and R₂₃ and R₂₄ are independently selected from hydrogen, alkyl, alkenyl, alkynyl, and aryl.

5. The bioresorbable CSA compound of claim 1, wherein at least one of R₁-R₁₈, preferably one, two or three of R₃, R₇, and R₁₂, has a structure selected from:
R₂₄R₂₃N—R₂₂—(C═O)—O—,
R₂₄R₂₃N—R₂₂—(C═O)—N—, and
R₂₄R₂₃N—R₂₂—O—
where R₂₂ is a substituted or unsubstituted alkyl and R₂₃ and R₂₄ are independently selected from hydrogen, alkyl, alkenyl, alkynyl, and aryl.

6. The bioresorbable CSA compound of claim 1, wherein:
R₁ through R₁₈ are independently selected from the group consisting of hydrogen, hydroxyl, substituted or unsubstituted (C₁-C₂₂)alkyl, substituted or unsubstituted (C₁-C₂₂) hydroxyalkyl, substituted or unsubstituted (C₁-C₂₂)alkyloxy-(C₁-C₂₂)alkyl, substituted or unsubstituted (C₁-C₂₂)alkylcarboxy-(C₁-C₂₂)alkyl, substituted or unsubstituted (C₅-C₂₅) terpenylcarboxy-(C₁-C₂₂)alkyl, substituted or unsubstituted (C₅-C25) terpenylcarbonyloxy-(C₁-C₂₂)alkyl, substituted or unsubstituted (C₅-C₂₅) terpenylcarboxamido-(C₁-C₂₂)alkyl, substituted or unsubstituted (C₅-C₂₅) terpenylamino-(C₁-C₂₂)alkyl, (C₅-C₂₅) terpenyloxyo-(C₁-C₂₂)alkyl, substituted or unsubstituted (C₁-C₂₂)alkylamino-(C₁-C₂₂)alkyl, substituted or unsubstituted (C₁-C₂₂)alkylamino-(C₁-C₂₂)alkylamino, substituted or unsubstituted (C₁-C₂₂)alkylamino-(C₁-C₂₂)alkylamino-(C₁-C₂₂)alkylamino, substituted or unsubstituted (C₁-C₂₂)aminoalkyl, substituted or unsubstituted aryl, substituted or unsubstituted arylamino-(C₁-C₂₂)alkyl, substituted or unsubstituted (C₁-C₂₂)haloalkyl, substituted or unsubstituted (C₂-C₆)alkenyl, substituted or unsubstituted (C₂-C₆)alkynyl, oxo, linking group attached to a second steroid, substituted or unsubstituted (C₁-C₂₂)aminoalkylurethanyl, substituted or unsubstituted (C₂-C₂₂)aminoalkenylurethanyl, substituted or unsubstituted (C₂-C₂₂) aminoalkynylurethanyl, and substituted or unsubstituted aminoarylurethanyl, substituted or unsubstituted (C₁-C₂₂)aminoalkyloxy, substituted or unsubstituted (C₁-C₂₂)aminoalkylcarboxy, substituted or unsubstituted (C₁-C₂₂)aminoalkyloxy-(C₁-C₂₂)alkyl, substituted or unsubstituted (C₁-C₂₂)aminoalkyl-aminocarbonyl, substituted or unsubstituted (C₁-C₂₂)aminoalkylcarboxamido, substituted or unsubstituted di (C₁-C₂₂)alkylamino-(C₁-C₂₂)alkyl, H₂N—HC (Q₅)-(C═O)—O—, H₂N—HC (Q₅)-(C═O)—NH—, substituted or unsubstituted (C₁-C₂₂)azidoalkyloxy, substituted or unsubstituted (C₁-C₂₂) cyanoalkyloxy, P.G.-HN—HC (Q₅)-(C═O)—O—, substituted or unsubstituted (C₁-C₂₂) guanidinoalkyloxy, substituted or unsubstituted quaternary ammonium (C₁-C₂₂)alkylcarboxy, and substituted or unsubstituted (C₁-C₂₂) guanidinoalkylcarboxy, where Q₅is a side chain of an amino acid (including a side chain of glycine, i.e., H), and P.G. is an amino protecting group; and
R₅, R₈, R₉, R₁₀, R₁₃, R₁₄ and R₁₇ are independently deleted when one of rings A, B, C, or D is unsaturated so as to complete the valency of the carbon atom at that site,
provided that at least one of Ri through R₄, R₆, R₇, R₁₁, R₁₂, R₁₅, R₁₆, and R₁₈, preferably R₁₈, includes a glyceride of a (C₂-C₂₂) fatty acid attached to the sterol backbone by an ester linkage, and
provided that at least one of Ri through R₄, R₆, R₇, R₁₁, R₁₂, R₁₅, R₁₆, and R₁₈, preferably one, two or three of R₃, R₇, and R₁₂, includes an amino acid attached to the sterol backbone by an ester linkage or amide linkage.

7. The bioresorbable CSA compound of claim 1, wherein
R₁₈ includes a mono-or diglyceride of a (C₂-C₂₂) fatty acid attached to the sterol backbone at the C24 position by an ester linkage.

8. The bioresorbable CSA compound of claim 1, wherein two or three of R₃, R₇ and R₁₂ are an amino acid attached to the sterol backbone by an ester linkage.

9. The bioresorbable CSA compound claim 1, wherein the fatty acid is selected from the group consisting of butyric acid, caproic acid, caprylic acid, capric acid, lauric acid, myristic acid, palmitic acid, stearic acid, and combinations thereof.

10. The bioresorbable CSA compound of claim 1, wherein the amino acid is selected from the group consisting of D-alanine, L-alanine, beta-alanine, L-asparagine, D-aspartic acid, L-aspartic acid, L-cysteine, L-glutamic acid, L-glutamine, glycine, L-proline, D-serine, L-serine, L-tyrosine, L-histidine, L-isoleucine, L-leucine, L-lysine, D-methionine, L-methionine, L-phenylalanine, L-threonine, L-tryptophan, D-valine, L-valine, L-ornithine, L-arginine, hypusine, 2-aminoisobutyric acid, dehydroalanine, γ-aminobutyric acid, L-citrulline, α-ethyl-glycine, α-propyl-glycine, and L-norleucine:

11. The bioresorbable CSA compound of claim 1, wherein the amino acid comprises beta-alanine.

12. The bioresorbable CSA compound of claim 1, wherein R₁, R₂, R₄, R₅, R₆, R₈, R₉, R₁₀, R₁₁, R₁₃, R₁₄, R₁₅, R₁₆, and R₁₇ are independently selected from the group consisting of hydrogen and unsubstituted (C₁-C₆)alkyl.

13. The bioresorbable CSA compound of claim 1, wherein R₃, R₇, and R₁₂ comprise the same amino acid ester group.

14. The bioresorbable CSA compound of claim 1, wherein R₁₈ comprises a mixed diglyceride comprising an alkylcarboxy group and an amino alkylcarboxy group.

15. The bioresorbable CSA compound of claim 1, wherein the CSA compound is selected from the group consisting of: 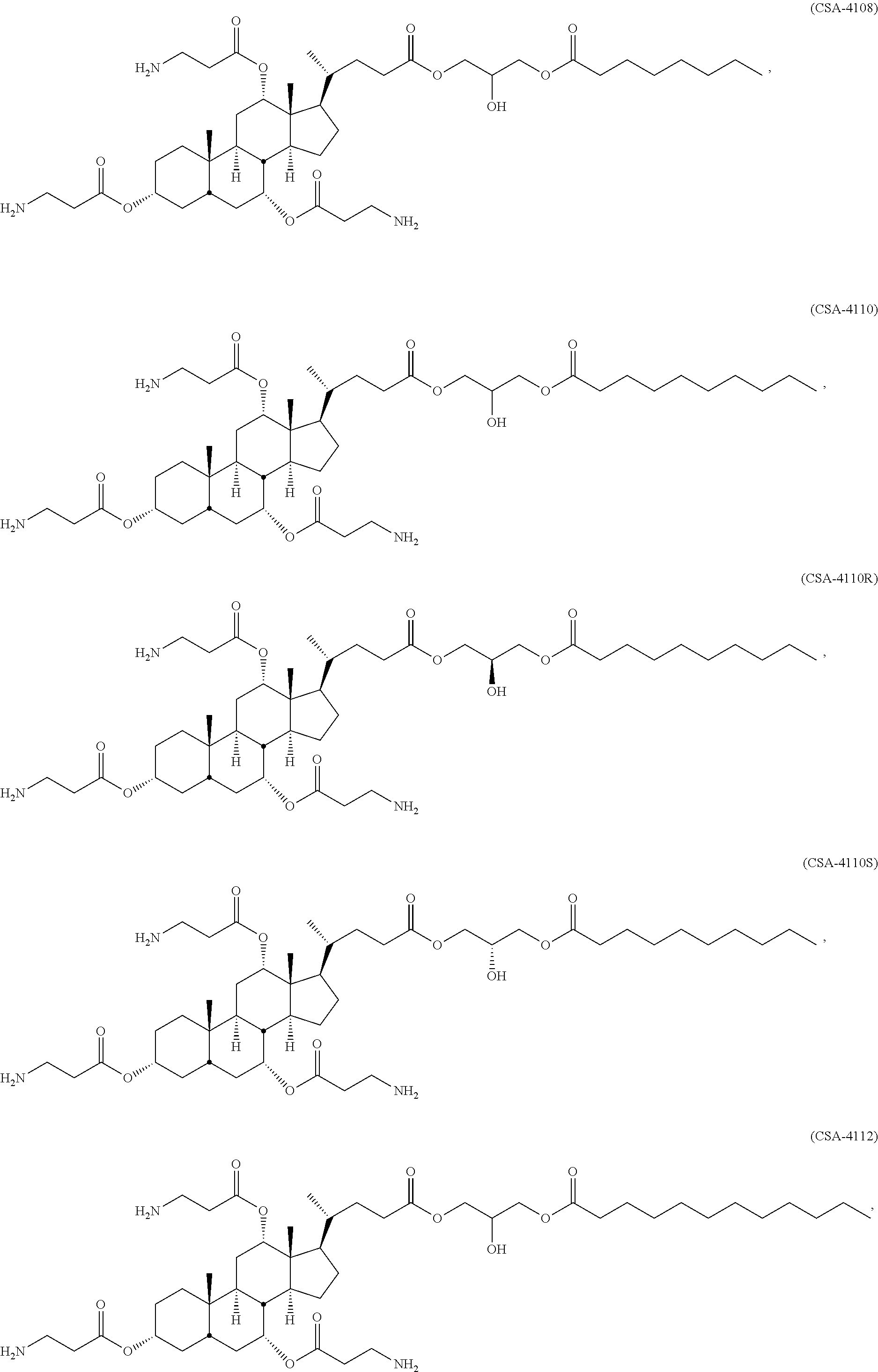 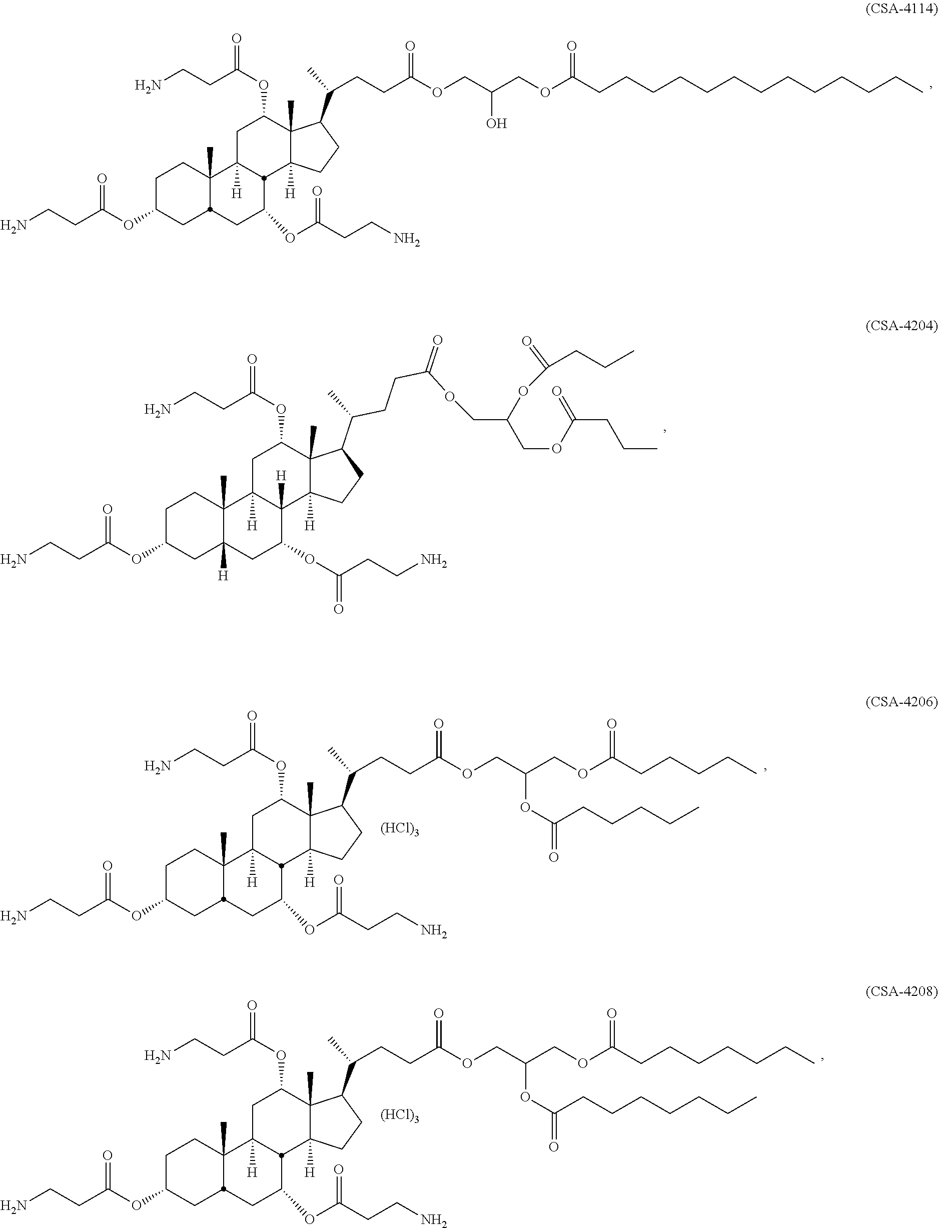 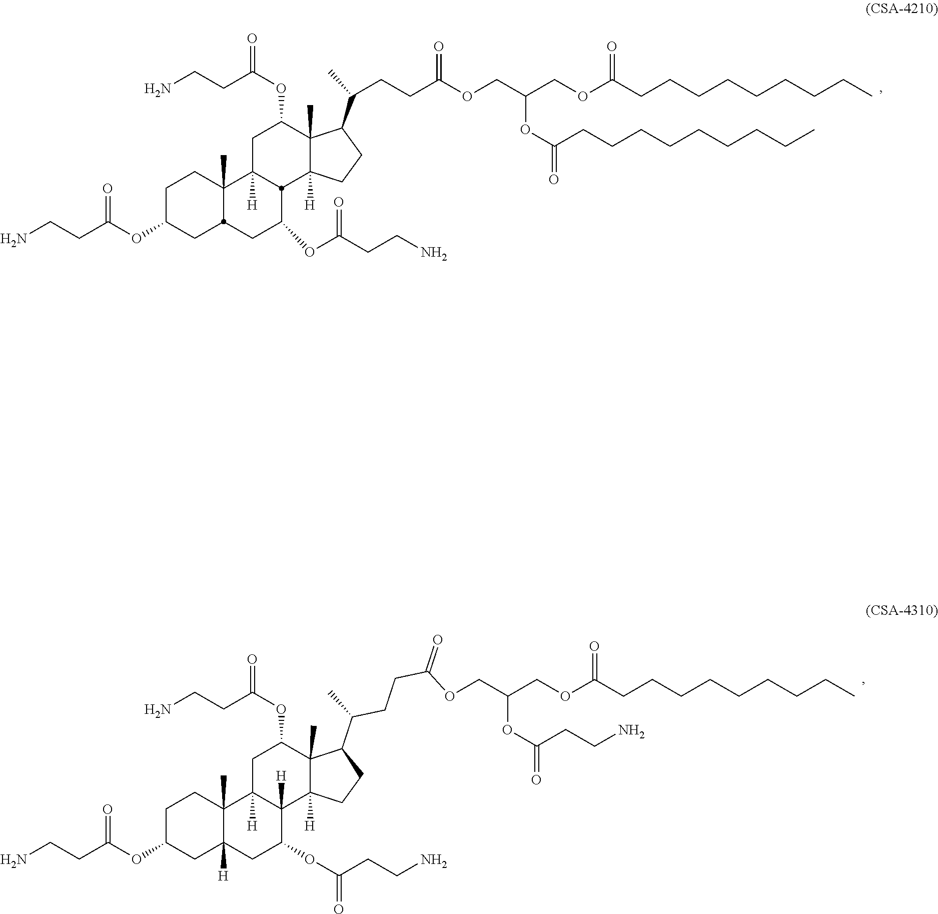
and salts thererof.

16. A pharmaceutical composition comprising a bioresorbable CSA compound of claim 1 and a pharmaceutically acceptable excipient selected from a carrier, solvent, stabilizer, adjuvant, and diluent.

17. A method of manufacturing a bioresorbable CSA compound of claim 1, comprising:
reacting cholic acid with protecting agent in a polar aprotic solvent in the presence of a base to form protected cholic acid having a protected carboxyl group at the C24 position;
reacting the protected cholic acid with an N-protected amino acid to form a first intermediate compound having an N-protected aminoalkylcarboxy ester group at one or more of the C3, C7 and C12 positions of the sterol backbone and the protected carboxyl group at the C24position;
deprotecting the carboxyl group of the first intermediate compound at the C24 position to form a second intermediate compound having an unprotected carboxyl group at the C24 position;
reacting the second intermediate compound with (i) a monoglyceride of a fatty acid, (ii) a diglyceride of a fatty acid, or (iii) a mixed diglyceride of a fatty acid and an N-protected amino acid, to form a third intermediate compound having a glyceride ester at the C24 position; and
deprotecting the N-protected aminoalkylcarboxy ester group(s) to yield the bioresorbable CSA compound or intermediate thereof.

18. The method of claim 17, wherein deprotecting the N-protected aminoalkylcarboxy group(s) includes treating with an acid (e.g., hydrochloric acid) to form an acid addition salt of the bioresorbable CSA compound or intermediate thereof.

19. The method of claim 18, further comprising neutralizing the acid addition salt of the bioresorbable CSA compound or intermediate thereof with a base and recovering a free base of the bioresorbable CSA compound or intermediate thereof.

20. The method of claim 19, further comprising acidifying the free base of the bioresorbable CSA compound or intermediate thereof with an acid to form a second acid addition salt of the bioresorbable CSA compound or intermediate thereof (e.g., 1,5-naphthalenedisulfonic acid di-addition salt).
